(19) United States
(12) Patent Application Publication
(10) Pub. No.: US 2002/0076409 A1
(43) Pub. Date: Jun. 20, 2002

(54) Method for treating cancer

(76) Inventors: Carl J. March, Bainbridge Island, WA (US); John D. Pluenneke, Parkville, MO (US); Larry F. O'Neal, Columbia, SC (US)

Correspondence Address: IMMUNEX CORPORATION, LAW DEPARTMENT, 51 UNIVERSITY STREET, SEATTLE, WA, 98101

(21) Appl. No.: 09/904,245

(22) Filed: Jul. 11, 2001

Related U.S. Application Data
(60) Provisional application No. 60/217,888, filed on Jul. 12, 2000.

(51) Int. Cl. A61K 39/395; A61K 38/17

(52) U.S. Cl. 424/145.1; 514/12

(57) ABSTRACT

Methods for treating cancer involve administering an interleukin-4 antagonist to a patient diagnosed with cancer. Suitable IL-4 antagonists include, but are not limited to, IL-4 receptors (IL-4R) such as a soluble human IL-4 receptor, antibodies that bind IL-4, antibodies that bind IL-4R, IL-4 muteins that bind to IL-4R but do not induce a biological response, molecules that inhibit IL-4-induced signal transduction, and other compounds that inhibit a biological effect that results from the binding of IL-4 to a cell surface IL-4R. Co-administration of an IL-4 antagonist and an immune stimulatory molecule is also contemplated. Particular antibodies provided herein include human monoclonal antibodies generated by procedures involving immunization of transgenic mice.

### CROSS-REFERENCE TO RELATED APPLICATION

This application claims the benefit of priority of U.S. Provisional Application Serial No. 60/217,888, filed Jul. 12, 2000, and which is incorporated herein in its entirety.

### BACKGROUND OF THE INVENTION

Interleukin-4 (IL-4), previously known as B cell stimulating factor, or BSF-1, was originally characterized by its ability to stimulate the proliferation of B cells in response to low concentrations of antibodies directed to surface immunoglobulin. However, since its discovery IL-4 has been shown to possess a far broader spectrum of biological activities, including growth co-stimulation of T cells, mast cells, granulocytes, megakaryocytes, and erythrocytes. In addition, IL-4 stimulates the proliferation of several IL-2- and IL-3-dependent cell lines, induces the expression of class II major histocompatibility complex molecules on resting B cells, and enhances the secretion of IgE and IgG1 isotypes by stimulated B cells. IL-4 is associated with a TH2-type immune response, being one of the cytokines secreted by TH2 cells.

Murine and human IL-4 have been identified and characterized, including cloning of IL-4 cDNAs and determination of the nucleotide and encoded amino acid sequences. (See Yokota et al., Proc. Natl. Acad. Sci. USA 83:5894, 1986; Noma et al., Nature 319:640, 1986; Grabstein et al., J. Exp. Med. 163:1405,1986; and U.S. Pat. No. 5,017,691.)

IL-4 binds to particular cell surface receptors, which results in transduction of a biological signal to cells such as various immune effector cells. IL-4 receptors are described, and DNA and amino acid sequence information presented, in Mosley et al., Cell 59:335-348, Oct. 20, 1989 (murine IL-4R); Idzerda et al., J. Exp. Med. 171:861-873, March 1990 (human IL-4R); and U.S. Pat. No. 5,599,905. The IL-4 receptor described in these publications is sometimes referred to as IL-4R-alpha.

Other proteins have been reported to be associated with IL-4R-alpha on some cell types, and to be components of multi-subunit IL-4 receptor complexes. One such subunit is IL-2R-gamma, also known as IL-2R-γ_{c} or γ_{c}. (See the discussion of IL-4R complexes in Sato et al., Current Opinion in Cell Biology, 6:174-179, 1994.) IL-4R-alpha has been reported to be a component of certain multi-subunit IL-13 receptor complexes (Zurawski et al., J. Biol. Chem. 270 (23), 13869, 1995; de Vries, J. Allergy Clin. Immunol. 102(2):165, August 1998; and Callard et al. Immunology Today, 17(3):108, March 1996).

IL-4 has been implicated in a number of disorders, examples of which are allergy and asthma. Studies of biological properties of IL-4 continue, in an effort to identify additional activities associated with this pleiotrophic cytokine, and to elucidate the role IL-4 may play in various biological processes and diseases.

### SUMMARY OF THE INVENTION

The present invention provides a method for treating oncologic conditions, comprising administering an interleukin-4 (IL-4) antagonist to a mammal afflicted with cancer. Also provided are compositions for use in such methods, comprising an effective amount of an IL-4 antagonist and a suitable diluent, excipient, or carrier.

The present invention further provides a method for treating oncologic conditions, comprising co-administering an IL-4 antagonist with a cytotoxic T-cell agonist, such as interferon-alpha, to a mammal afflicted with cancer. Alternatively, the antagonist is administered first, followed by subsequent administration of the cytotoxic T-cell agonist.

Other methods provided herein comprise contacting a cancer patient's bodily fluid that contains endogenous IL-4 with an IL-4 antagonist that binds IL-4, in an ex vivo procedure. The amount of IL-4 in the bodily fluid returned to the patient is thereby reduced. It is also provided that upon returning the fluid to the patient, a cytotoxic T-cell agonist is administered to the patient, for example interferon-alpha. The T-cell agonist can also be administered to the fluid prior to its return to the patient, for example, simultaneously with the IL-4 antagonist.

IL-4 antagonists include but are not limited to IL-4 receptors (IL-4R), antibodies that bind IL-4, antibodies that bind IL-4R, IL-4 muteins that bind to cell surface IL-4R but do not induce a biological response, molecules that inhibit IL-4-induced signal transduction, and other compounds that inhibit a biological effect that results from the binding of IL-4 to a cell surface IL-4R.

Cytotoxic T-cell agonists include but are not limited to interferon-alpha isotypes.

Examples of IL-4 receptors that may be employed as IL-4 antagonists are soluble forms of the human IL-4R of SEQ ID NO:2. Among the antibodies provided herein for use as IL-4 antagonists are human monoclonal antibodies generated by procedures involving immunization of transgenic mice. Such human antibodies may be raised against human IL-4R, for example. In particular embodiments, antibodies raised against IL-4R are also capable of inhibiting IL-13-induced biological activities.

### BRIEF DESCRIPTION OF THE DRAWINGS

FIGS. 1A to 1C depict the nucleotide sequence of the coding region of a human IL-4 receptor cDNA. The amino acid sequence encoded by the cDNA is presented as well. The cDNA clone was isolated from a cDNA library derived from a human T cell line T22. The encoded protein comprises (from N- to C-terminus) an N-terminal signal peptide, followed by an extracellular domain, a transmembrane region (underlined), and a cytoplasmic domain, as discussed further below. The DNA and amino acid sequences of FIGS. 1A to 1C are also presented in SEQ ID NOS:1 and 2, respectively.

FIGS. 2A to 2C depict targeted insertion of a neo cassette into the SmaI site of the μ1 exon. The construct was employed in generating transgenic mice, as described in Example 2. FIG. 2A is a schematic diagram of the genomic structure of the μ locus. The filled boxes represent the μ exons. FIG. 2B is a schematic diagram of the CmD targeting vector. The dotted lines denote those genomic μ sequences included in the construct. Plasmid sequences are not shown. FIG. 2C is a schematic diagram of the targeted μ locus in which the neo cassette has been inserted into μ1.

FIGS. 3A and 3B depict the nucleotide sequence of a vector designated pGP1k, as described in Example 3 below.

### DETAILED DESCRIPTION OF THE INVENTION

The present invention provides a method for treating cancer, comprising administering an IL-4 antagonist to a mammal, including a human, afflicted with cancer. Compositions for use in such methods also are provided, comprising at least one IL-4 antagonist and a diluent, carrier, or excipient.

The present invention further provides a method for treating oncologic conditions, comprising co-administering an IL-4 antagonist with a cytotoxic T-cell agonist to a mammal afflicted with cancer, wherein an effective amount of the antagonist is administered simultaneously with an effective amount of the cytotoxic T-cell agonist. Alternatively, the IL-4 antagonist is administered first, followed by subsequent administration of an effective amount of the cytotoxic T-cell agonist.

The present invention further provides a method for reversing non-responsiveness to interferon-alpha therapy by administration of an effective dose of an antagonist of IL-4. More particularly, the invention provides a method for enhancing responsiveness, or restoring a human cancer patients responsiveness to interferon-alpha treatment by administration of an effective dose of an antagonist of IL-4, such that interferon-alpha treatment stimulates a cytotoxic T-cell response.

IL-4 antagonists that may be employed include those compounds that inhibit a biological activity of IL-4. Biological activity(ies) of IL-4 that are inhibited by an antagonist in accordance with methods provided herein are activities that play a role in the particular oncologic condition to be treated. Oncologic conditions in which IL-4 plays a role are thus alleviated.

Suitable antagonists include, but are not limited to, IL-4 receptors, antibodies that bind IL-4, antibodies that bind IL-4R, IL-4 muteins that bind to IL-4R but do not induce biological responses, molecules that inhibit IL-4-induced signal transduction, and other compounds that inhibit a biological effect that results from the binding of IL-4 to a cell surface IL-4R. Examples of such IL-4 antagonists are described in more detail below. The antagonists may be prepared and purified by conventional procedures, as discussed below. In particular embodiments, the IL-4 antagonists also function as IL-13 antagonists. Antibodies that are raised against IL-4R may be screened to identify those that additionally inhibit IL-13-induced biological activity, as discussed below.

Indications

The present invention provides methods comprising administering an IL-4 antagonist to a patient who has cancer. Methods for treating a mammal, including a human patient, who has cancer, comprise administering an IL-4 antagonist to the mammal in vivo, or otherwise contacting endogenous IL-4 with an IL-4 antagonist, e.g., in an ex vivo procedure. The invention also provides methods comprising co-administration of an IL-4 antagonist with a cytotoxic T-cell agonist, such as a cytokine, e.g., interferon-alpha. Alternatively, the invention provides methods comprising co-administration of an IL-4 antagonist followed by administration of a cytotoxic T-cell agonist, e.g., interferon-alpha.

Among the conditions that may be treated in accordance with such methods are oncologic conditions (cancer and cancer-related conditions) that are caused or exacerbated, directly or indirectly, by IL-4. Other factors or cytokines also may play a role in such conditions, but IL-4 induces or mediates the condition to some degree, i.e., at least in part.

Forms of cancer treatable in accordance with methods provided herein include but are not limited to leukemia, lymphoma, carcinoma, blastoma, sarcoma, and melanoma. Particular examples of such cancers include but are not limited to: cutaneous T-cell leukemia (e.g., Sezary syndrome), chronic or acute lymphoblastic leukemia, chronic or acute lymphocytic leukemia, hairy cell leukemia, Hodgkin's lymphoma (Hodgkin's disease), non-Hodgkin's lymphoma (e.g., large cell anaplastic lymphoma), peripheral T-cell lymphoma, small lymphocytic lymphoma, follicular lymphoma, histiocytic lymphoma, diffuse aggressive lymphoma, colon carcinoma, breast carcinoma, lung carcinoma (small-cell lung cancer and non-small cell lung cancer), adenocarcinoma, nasopharyngeal carcinoma, squamous cell carcinoma, glioblastoma multiforme, rhabdomyosarcoma, and osteosarcoma. Further examples of types of cancer include head and neck cancers, colorectal cancer, other gastrointestinal cancers, glioma, neuroblastoma, cervical cancer, endometrial cancer, and cancer of the pancreas, thyroid, stomach, colon, bladder, skin, breast, prostate, ovary, kidney, or liver.

Further examples of cancers to be treated by the methods and compositions of the invention include but are not limited to renal cancer including renal carcinoma; bronchogenic cancers e.g., bronchogenic carcinomas; and metastases localized in lung, with one example being pulmonary metastases from melanoma.

One particular embodiment of the invention is directed to a method of treating a hematologic malignancy, comprising administering an IL-4 antagonist to a mammal, such as a human, afflicted with such a malignancy. For example, an IL-4 antagonist is administered to a human diagnosed with a B-cell or T-cell malignancy or other malignant lymphoproliferative disorder.

Examples of low grade B-cell malignancies include: low grade non-Hodgkin's disease and low grade B lymphomas; chronic lymphocytic leukemia (CLL), including B-type chronic lymphocytic leukemia (B-CLL) and small lymphocytic lymphoma; acute myeloid leukemia (AML, also known as acute myelogenous leukemia); chronic myeloid leukemia (CML, also known as chronic myelogenous leukemia); and myeloma, including multiple myeloma.

Another B-cell lymphoma which can be treated by the methods and compositions of the invention is Hodgkin's lymphoma (Hodgkin's disease) which is categorized as either nodular lymphocyte-predominant Hodgkin's lymphoma or classical Hodgkin's lymphoma. The latter classification includes nodular sclerosis Hodgkin's lymphoma, lymphocyte-rich classical Hodgkin's lymphoma, mixed cellularity Hodgkin's lymphoma and lymphocyte depletion Hodgkin's lymphoma.

Additional B-cell neoplasms and precursor B-cell neoplasms include: precursor B-acute lymphoblastic leukemia/lymphoblastic lymphoma (B-ALL, LBL) and peripheral B-cell neoplasms including B-cell prolymphocytic leukemia, lymphoplasmacytic lymphoma/immunocytoma, mantle cell lymphoma, follicular lymphoma, extranodal marginal zone B-cell lymphoma of MALT type, nodal marginal zone B-cell lymphoma (+/−monocytoid B-cells), splenic marginal zone lymphoma (+/−villous lymphocytes), plasmacytoma/plasma cell myeloma, diffuse large B-cell lymphoma, and Burkitt's lymphoma.

T-cell and putative NK-cell neoplasms as well as precursor T-cell neoplasms include:

precursor T-acute lymphoblastic leukemia/lymphoblastic lymphomas (T-ALL, LBL), and peripheral T-cell and NK-cell neoplasms, such as T-cell chronic lymphocytic leukemia/prolymphocytic leukemia, T-cell granular lymphocytic leukemia, peripheral T-cell lymphoma (not otherwise characterized), hepatosplenic gamma/delta T-cell lymphoma, subcutaneous panniculitis-like T-cell lymphoma, angioimmunoblastic T-cell lymphoma, extranodal T-/NK-cell lymphoma (nasal type), enteropathy-type intestinal T-cell lymphoma, adult T-cell lymphoma/leukemia (HTLV 1+), anaplastic large cell lymphoma, primary systemic type, anaplastic large cell lymphoma (primary cutaneous type), and aggressive NK-cell leukemia.

Another cancer includes cutaneous T-cell lymphoma (CTCL). CTCL has been classified further into groups as follows: Mycosis Fungoides, Sezary syndrome, pagetoid reticulosis, CD30 positive cutaneous large T-cell lymphoma, lymphomatoid papulosis, and CD30 negative cutaneous large T-cell lymphoma. In a particular embodiment, the methods and compositions of the invention are used to treat Sezary syndrome.

Treatment according to the present invention also serves to alleviate cancer-associated conditions. Examples of such conditions include but are not limited to cachexia, fatigue, asthenia, paraneoplastic syndrome of cachexia, and hypercalcemia. Additional examples of such conditions include lymphadenopathy, erythroderma, eczematous patch/plaques, cutaneous lesions and/or cutaneous ulceration.

Biological activities of IL-4 are mediated through binding to specific cell surface receptors. IL-4-induced oncologic conditions include those arising from biological responses that result from the binding of IL-4 to native IL-4 receptors on cells, or which may be inhibited or suppressed by preventing IL-4 from binding to an IL-4 receptor. Conditions that may be treated include, but are not limited to, oncologic conditions characterized by abnormal or excess expression of IL-4, or by an abnormal host response to IL-4 production. Further examples are conditions in which IL-4-induced antibody production or proliferation or influx of a particular cell type plays a role. IL-4-induced disorders include those in which IL-4 induces upregulation of IL-4 receptors or enhanced production of another protein that plays a role in a disease (e.g., another cytokine).

IL-4 is implicated as playing a role in promoting growth, proliferation, survival, development, or differentiation of some types of cancer cells. IL-4 antagonists administered in accordance with the present invention inhibit IL-4-induced growth, proliferation, survival, development, or differentiation of cancer cells. In one illustrative embodiment, the IL-4 antagonist reduces proliferation of malignant B cells.

IL-4 antagonists may be employed to shift the patient's immune response toward a cytotoxic T-cell-mediated immune response (e.g., characterized by a TH1-type immune phenotype) rather than an antibody-mediated immune response (e.g., characterized by a TH2-type immune phenotype). Distinct subsets of helper T-cells play an important role in regulating these two arms of the immune response. The helper T-cell subsets can be distinguished by properties that include the specific array of cytokines secreted by the cells. Cytokines associated with a TH1-type immune response (i.e., cytokines secreted by T helper cells of type 1, TH1 cells) include interferon (IFN)-gamma, IL-2, and tumor necrosis factor (TNF)-beta. Cytokines associated with a TH2-type immune response (i.e., secreted by T-helper cells of type 2, TH2 cells) include IL-4, IL-5 and IL-10.

The use of IL-4 antagonists is advantageous at times when promoting a TH1-type immune response would be beneficial to the patient. IL-4 antagonists may be administered to promote a T-cell mediated immune response, for example to a tumor or malignancy. The IL-4 antagonist may be said to reduce proliferation of TH2 cells, to suppress a TH2 response, to shift the immune response toward a TH1 response, to favor a TH1-type immune response, or to promote synthesis of TH1-type cytokines by T-cells.

The use of an antagonist to IL-4 is also advantageous when promoting the development of TH1 -type immune cells in a patient with a T-cell lymphoma, particularly when the lymphoma cells have a TH2-type immune phenotype, as can be the case in cutaneous T-cell lymphoma (CTCL) such as Sezary syndrome.

In a particular example, when a Sezary syndrome patient is diagnosed with T-lymphoma that is derived from T-cells with a TH2-type immune phenotype, IL-4 antagonists can be used to promote a shift away from the TH2-type immune cells toward a TH1-type immune phenotype. Further, the administration of an IL-4 antagonist can be used to stimulate production of TH1-type cells in a Sezary syndrome patient, which are then stimulated to attack the cancer cells, for example, by co-administration of interferon-alpha resulting in the activation of the TH1-type immune response.

An IL-4 antagonist may be tested for the ability to promote development of a TH1 immune phenotype and/or response using conventional assay techniques, such as cytokine profile or cytokine production assays. For example, cytokines secreted into the culture supernatant by CD8+ and/or CD4+ T cell lines, in response to antigen, may be quantitatively determined in in vitro assays. Such assays are conducted in the presence and absence of an IL-4 antagonist, to confirm the ability of the IL-4 antagonist to increase the secretion of TH1-type cytokines.

IL-4 antagonists are especially advantageous when promoting a T-cell mediated immune response (a TH1-type immune response) against a tumor or malignancy would be beneficial. IL-4 antagonists may be employed in immunotherapy of cancer, either alone or in combination with other agents that enhance or augment a patient's cellular immune response against cancer. For example, an IL-4 antagonist is co-administered with interferon-alpha to a cancer patient in need thereof. Alternatively, the IL-4 antagonist is administered first, and subsequently, interferon-alpha is administered to a cancer patient in need thereof. In this manner, normal TH1 immune cells are stimulated to proliferate by the IL-4 antagonist and the interferon-alpha subsequently augments the cell mediated immune response.

In one embodiment, an IL-4 antagonist is added to a therapeutic regimen involving adoptive immunotherapy. In one example provided herein, T lymphocytes are extracted from a cancer patient, then activated and expanded in vitro to generate anti-tumor effector cells directed against the patient's tumor cells, and finally an IL-4 antagonist is co-administered to the patient along with the thus-generated anti-tumor effector cells (anti-tumor T-cells). Additionally, a TH1 immune cell agonist can also be administered, for example, interferon-alpha, which then serves to activate the anti-tumor effector cells.

IL-4 antagonists also find use as adjuvants for cancer vaccines, including vaccines used to prevent or to treat cancer. Such vaccines may contain or be derived from tumor antigens. While not being limited to a particular mechanism of action, an IL-4 antagonist co-administered with an anti-cancer vaccine may enhance development of a TH1-type immune response toward a cancer antigen. The antagonist may be administered simultaneously with the vaccine, or may be administered separately one or more times, during the course of a therapeutic or prophylactic regimen of vaccine administration.

IL-4 antagonists may be administered to inhibit angiogenesis in cancer patients, including but not limited to patients with solid tumors. Inhibiting angiogenesis (neovascularization) associated with tumor tissue limits tumor growth or metastases. One example of a therapeutic method provided herein comprises subcutaneous administration of an IL-4 antagonist to a patient who has a solid tumor.

IL-4 has been implicated as playing a role in suppressing apoptosis of tumor cells in vivo. Administration of IL-4 antagonists in accordance with the present invention inhibits the apoptosis-suppressing activity of IL-4, thereby rendering the cancer cells more susceptible to apoptosis, and allowing apoptosis of cancer cells to occur. The resulting cancer cell apoptosis may occur naturally, e.g. induced by endogenous molecules and mechanisms, or may be induced by an agent such as an anti-cancer drug. Suppression of apoptosis has been linked to development of drug resistance in cancer cells during chemotherapy. IL-4 antagonists may be employed to promote apoptosis of cancer cells that have developed resistance to chemotherapy drug(s). In one embodiment, an IL-4 antagonist is administered to a patient afflicted with a low-grade lymphoma, such as a low-grade B-lymphoma, wherein the antagonist is administered in an amount effective in increasing apoptosis of the lymphoma cells. Other embodiments are directed to a method for increasing apoptosis of chronic lymphocytic leukemia (CLL) or acute lymphocytic leukemia (ALL) cells or B chronic lymphocytic leukemia (B-CLL) cells comprises administering an IL-4 antagonist to a CLL, ALL or B-CLL patient. In yet other embodiments, the invention provides methods of increasing apoptosis in cells associated with Sezary syndrome, comprising administering an antagonist of IL-4 to a Sezary syndrome patient in need thereof.

Administering an IL-4 antagonist may result in upregulation (increased expression) of another molecule, e.g., a cell surface protein, that contributes to death of cancer cells. One example involves upregulation of CD40 ligand (CD40-L) on tumor cells, following administration of an IL-4 antagonist. The CD40-L expression renders tumor cells more immunogenic toward various immune system cells, including T-cells and dendritic cells, thus increasing the patient's immune response directed against the tumor. Thus, IL-4 antagonists indirectly (via upregulating CD40-L) enhance development of cytotoxic T-cells directed against the patient's tumor, and enhance an anti-tumor immune response mediated by dendritic cells. In one embodiment, the tumor cells are B-CLL cells.

IL-4 Antagonists

IL-4 antagonists that may be employed in accordance with the present invention include compounds that inhibit a biological activity of IL-4. The IL-4-induced biological activities to be inhibited by the methods provided herein are activities that directly or indirectly play a role in the condition to be treated.

Examples of IL-4 antagonists include, but are not limited to, IL-4 receptors (IL-4R), antibodies, other IL-4-binding molecules, and IL-4 muteins as discussed further below. The antibodies may bind IL-4 or may bind an IL-4 receptor, for example.

Antagonists that bind IL-4 include but are not limited to IL-4 receptors and anti-IL-4 antibodies. Endogenous IL-4 that becomes bound to such an antagonist is thereby prevented from binding its natural receptor on cell surfaces in vivo, and thus does not induce IL-4-mediated biological activities.

Different types of antagonists may act at different sites or by different mechanisms of action. Examples include but are not limited to antagonists that interfere with binding of IL-4 to cell surface receptors or that inhibit signal transduction. The site of action may be intracellular (e.g., by interfering with an intracellular signaling cascade), on a cell surface, extracellular, or (in ex vivo procedures) outside the patient's body, for example. Antagonists that act by interfering with the interaction of IL-4 with IL-4R may bind to either IL-4 or to the receptor. An antagonist need not completely inhibit an IL-4 induced activity to find use in the present invention; rather, antagonists that reduce a particular activity of IL-4 are contemplated for use as well.

The above-presented discussions of particular mechanisms of action for IL-4 antagonists in treating particular conditions are illustrative only, and the methods presented herein are not bound thereby. The mechanisms of action by which IL-4 antagonists ameliorate oncologic conditions are not limited to those discussed above.

An IL-4 antagonist may reduce the amount of active IL-4 at a particular site within the body that is involved in an oncologic disorder. Antagonists that bind IL-4 such that it no longer can bind to endogenous cellular receptors functionally reduce the amount of active IL-4 available for inducing biological responses.

An IL-4 antagonist may alleviate a disorder by reducing the amount of free endogenous IL-4 that is circulating in the body, e.g., in the bloodstream or in a particular tissue. When the action of IL-4 on such tissue plays a role in pathogenesis of the disease, the antagonist serves to block action of IL-4 in the tissue, thereby alleviating the disorder. In a further example, antagonists may inhibit IL-4-induced recruitment of cells to a site or tissue within the body, wherein such recruitment plays a role in causing or exacerbating an oncologic condition.

Combinations of two or more antagonists may be employed in methods and compositions of the present invention. Examples of suitable IL-4 antagonists are as follows.

IL-4 Receptor

A preferred IL-4 antagonist is an IL-4 receptor (IL-4R). When administered in vivo, IL-4R polypeptides circulate in the body and bind to circulating endogenous IL-4 molecules, preventing interaction of IL-4 with endogenous cell surface IL-4 receptors, thus inhibiting transduction of IL-4-induced biological signals.

IL-4 receptors are described in U.S. Pat. No. 5,599,905; Idzerda et al., J. Exp. Med. 171:861-873, March 1990 (human IL-4R); and Mosley et al., Cell 59:335-348, October 20, 1989 (murine IL-4R); each of which is hereby incorporated by reference. The protein described in those three references is sometimes referred to in the scientific literature as IL-4R-alpha. Unless otherwise specified, the terms “IL-4R” and “IL-4 receptor” as used herein encompass this protein in various forms that are capable of functioning as IL-4 antagonists, including but not limited to soluble fragments, fusion proteins, oligomers, and variants that are capable of binding IL-4, as described in more detail below.

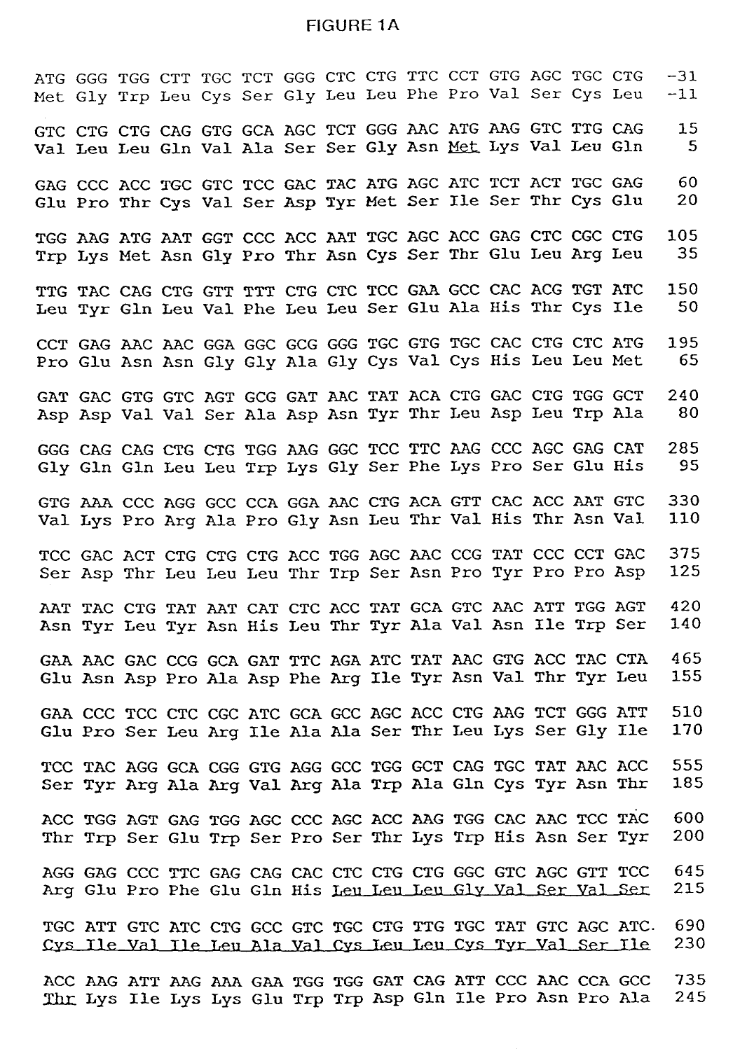

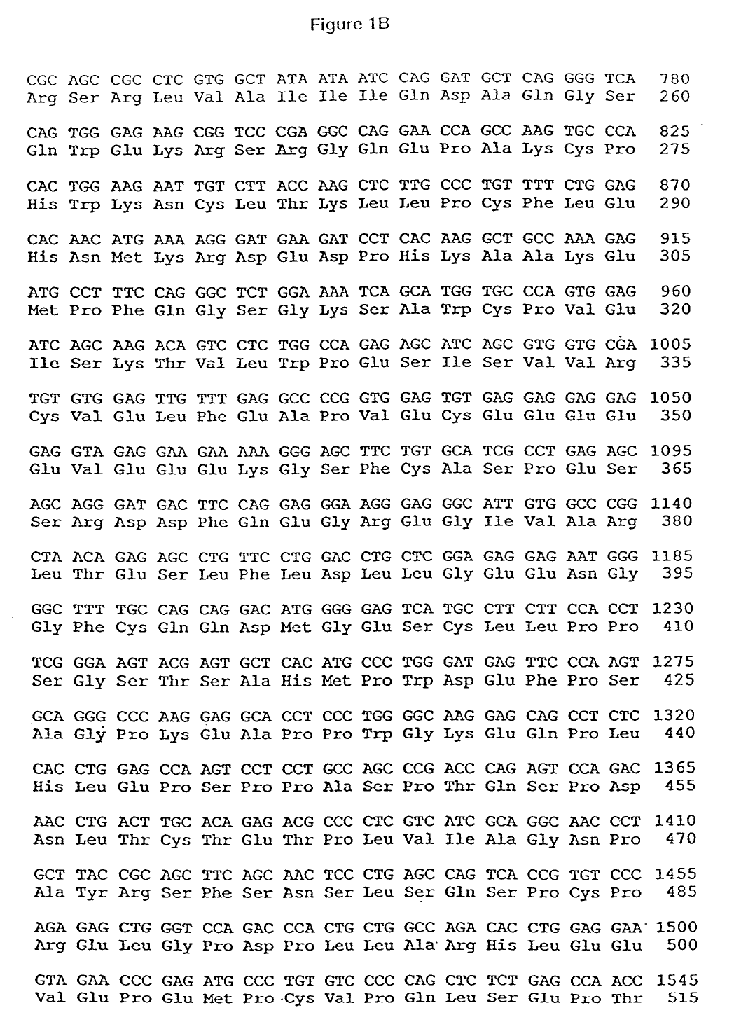

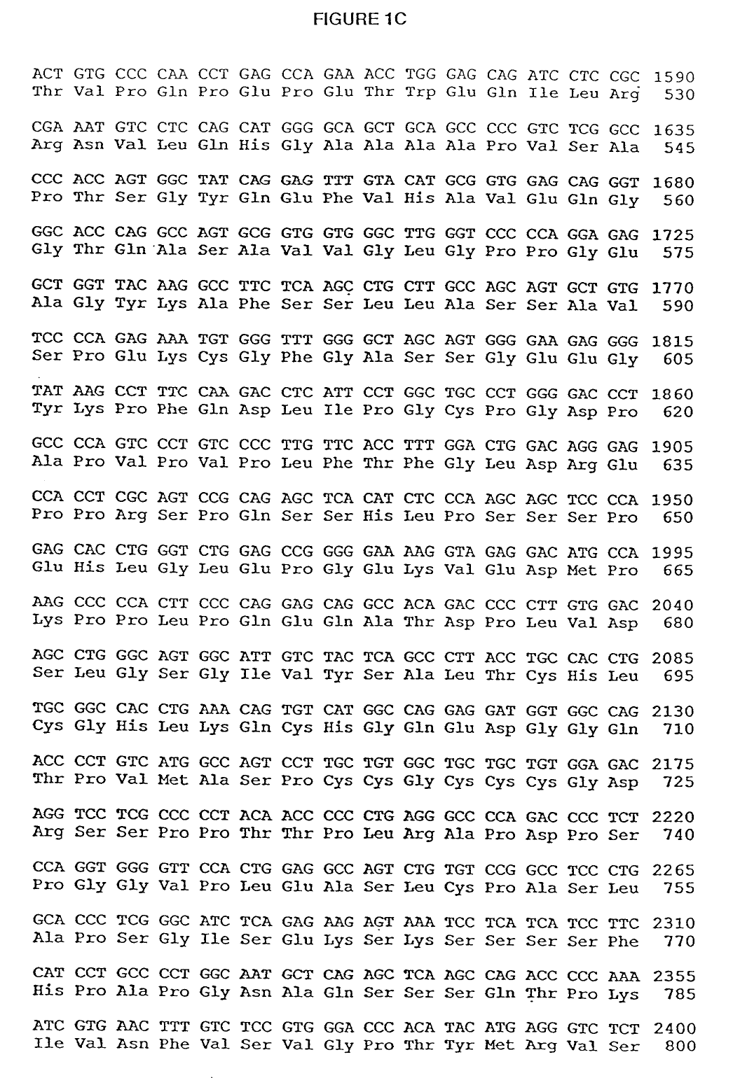

The nucleotide sequence of a human IL-4R cDNA, and the amino acid sequence encoded thereby, are set forth in FIGS. 1A-1C. The cDNA clone was isolated from a cDNA library derived from a CD4⁺/CD8⁻ human T cell clone designated T22, as described in Idzerda et al., J. Exp. Med., 171:861, March 1990, and in U.S. Pat. No. 5,599,905, which are hereby incorporated by reference in their entirety. The DNA and amino acid sequences of FIGS. 1A-1C are presented in SEQ ID NO:1 and SEQ ID NO:2, respectively.

The encoded human IL-4R protein comprises (from N- to C-terminus) an N-terminal signal peptide, followed by an extracellular domain, a transmembrane region, and a cytoplasmic domain. The transmembrane region, which is underlined in FIG. 1A, corresponds to amino acids 208 through 231. The cytoplasmic domain comprises amino acids 232 through 800.

A signal peptide includes amino acids −25 to −1 of SEQ ID NO:2. An alternative signal peptide cleavage site occurs between residues −3 and −2 of SEQ ID NO:2, such that the signal peptide corresponds to residues −25 through −3.

As is recognized in the pertinent field, the signal peptide cleavage site for a given protein may vary according to such factors as the particular expression system (especially the host cells) in which the protein is expressed. The exact boundaries of the signal peptide, and thus the extracellular domain, of a given recombinant protein thus may depend on the expression system employed. Further, the signal peptide may be cleaved at more than one position, generating more than one species of polypeptide in a preparation of recombinant protein.

In one embodiment, in which an expression vector comprises DNA encoding amino acids −25 through 207 of SEQ ID NO:2, the expressed recombinant IL-4R includes two species of mature soluble human IL-4R. The expressed polypeptides include a major species corresponding to amino acids −2 to 207 and a minor species corresponding to amino acids 1 to 207 of SEQ ID NO:2. Two alternate forms of the extracellular domain of human IL-4R thus correspond to residues −2 to 207 and 1 to 207 of SEQ ID NO:2. The term “mature” refers to a protein in a form lacking a signal peptide or leader sequence, as is understood in the pertinent art.

Among the IL-4 receptors suitable for use herein are IL-4R fragments. Truncated IL-4R polypeptides may occur naturally, e.g., as a result of proteolytic cleavage, post-translational processing, or alternative splicing of mRNA. Alternatively, fragments may be constructed by deleting terminal or internal portions of an IL-4R sequence, e.g., via recombinant DNA technology. Fragments that retain the ability to bind IL-4 may be identified in conventional binding assays. Such fragments may be soluble fragments, as discussed below.

In a preferred embodiment of the invention, the antagonist comprises a soluble form of the IL-4R. A soluble IL-4 receptor is a polypeptide that is secreted from the cell in which it is expressed, rather than being retained on the cell surface. The full length human IL-4R protein of SEQ ID NO:2 is a transmembrane protein, which, as described above, comprises an N-terminal signal peptide, followed by an extracellular domain, a transmembrane region, and a C-terminal cytoplasmic domain. Soluble IL-4R polypeptides lack the transmembrane region that would cause retention on the cell, and the soluble polypeptides consequently are secreted into the culture medium. The transmembrane region and intracellular domain of IL-4R may be deleted or substituted with hydrophilic residues to facilitate secretion of the receptor into the cell culture medium.

Particular embodiments of soluble IL-4R polypeptides lack the transmembrane region but comprise the extracellular domain (the complete extracellular domain or a fragment thereof that is capable of binding IL-4). As one option, the polypeptide comprises all or part of the cytoplasmic domain, as well as the extracellular domain (or fragment of the extracellular domain), but lacks the transmembrane region.

Examples of soluble human IL-4R polypeptides include, but are not limited to, polypeptides comprising amino acids residues x to y of SEQ ID NO:2, wherein x represents 1 or −2 and y represents an integer from 197 to 207. Preferred embodiments include polypeptides comprising residues 1 to 207 or −2 to 207 of SEQ ID NO:2.

A protein preparation administered as an IL-4 antagonist may comprise more than one form of IL-4R. For example, the preparation may comprise polypeptide molecules consisting of amino acids 1 to 207 of SEQ ID NO:2, as well as polypeptides consisting of amino acids −2 to 207 of SEQ ID NO:2.

IL-4R polypeptides arising from alternative mRNA constructs, e.g., which can be attributed to different mRNA splicing events following transcription, and which yield polypeptide translates capable of binding IL-4, are among the IL-4R polypeptides disclosed herein. Such alternatively spliced mRNAs may give rise to soluble polypeptides.

Further examples of IL-4 receptors that may be employed in the methods provided herein are variants having amino acid sequences which are substantially similar to the native interleukin-4 receptor amino acid sequence of SEQ ID NO:2, or fragments thereof. Variant IL-4 receptor polypeptides that are capable of functioning as IL-4 antagonists may be employed in the methods of the present invention.

Any of a number of conventional assay techniques may be employed to confirm that a given form of IL-4R (e.g., an IL-4R fragment or variant) functions as an IL-4 antagonist. Examples include binding assays or assays that test the ability of a given IL-4R polypeptide to inhibit transduction of an IL-4-induced biological signal. Examples of suitable in vitro assays are described below.

“Substantially similar” IL-4 receptors include those having amino acid or nucleic acid sequences that vary from a native sequence by one or more substitutions, deletions, or additions, but retain a desired biological activity of the IL-4R protein. Examples of nucleic acid molecules encoding IL-4 receptors include, but are not limited to: (a) DNA derived from the coding region of a native mammalian IL-4R gene; (b) DNA that is capable of hybridization to a DNA defined in (a) under moderately stringent conditions and which encodes an IL-4R having a biological activity of a native IL-4R; or (c) DNA that is degenerate as a result of the genetic code to a DNA defined in (a) or (b) and which encodes an IL-4R having a biological activity of a native IL-4R. Due to code degeneracy, there can be considerable variation in nucleotide sequences encoding the same amino acid sequence.

Variants may be naturally occurring, such as allelic variants or those arising from alternative splicing of mRNA. Alternatively, variants may be prepared by such well known techniques as in vitro mutagenesis.

A variant sequence identified by Idzerda et al., supra, comprises a GTC codon encoding the amino acid valine (Val) at position 50, instead of isoleucine (Ile). The variant sequence is otherwise identical to the sequence of SEQ ID NOS:1 and 2. IL-4R fragments, such as soluble fragments, comprising Val at position 50 are provided herein.

In particular embodiments, an IL-4 receptor DNA or amino acid sequence is at least 80 percent identical to the sequence of a native IL-4R. Preferably, an IL-4R DNA or polypeptide comprises a sequence that is at least 90 percent identical to a native IL-4R DNA or amino acid sequence. One example is a human IL-4R comprising an amino acid sequence that is at least 80 percent identical to the sequence presented in SEQ ID NO:2. Another example is a soluble IL-4R comprising an amino acid sequence at least 80 percent identical to the sequence of the extracellular domain of human IL-4R. Further examples are polypeptides comprising amino acid sequences that are at least 90 percent identical to the sequence presented in SEQ ID NO:2, or a fragment thereof. In a particular embodiment, the polypeptide comprises no more than 10 amino acid substitutions. IL-4R polypeptides that retain the ability to bind IL-4 may be identified in conventional binding assays.

Percent similarity or percent identity may be determined, for example, by comparing DNA or amino acid sequence information using the GAP computer program, version 6.0, available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math. 2:482, 1981). Briefly, the GAP program defines similarity as the number of aligned symbols (i.e., nucleotides or amino acids) which are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, ed., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps.

IL-4R polypeptides that vary from native proteins but possess a desired property may be constructed by, for example, substituting or deleting residues not needed for the particular biological activity. Substitutions may be conservative substitutions, such that a desired biological property of the protein is retained. Amino acids may be replaced with residues having similar physicochemical characteristics.

Cysteine residues can be deleted or replaced with other amino acids to prevent formation of incorrect intramolecular disulfide bridges upon renaturation. Other alterations of a native sequence involve modification of adjacent dibasic amino acid residues, to enhance expression in yeast host cells in which KEX2 protease activity is present.

The present invention also includes IL-4R with or without associated native-pattern glycosylation. The glycosylation pattern may vary according to the type of host cells in which the protein is produced. Another option is inactivation of N-glycosylation sites by site-specific mutagenesis. N-glycosylation sites in eukaryotic proteins are characterized by the amino acid triplet Asn-A₁-Z, where A₁ is any amino acid except Pro, and Z is Ser or Thr. In this sequence, asparagine provides a side chain amino group for covalent attachment of carbohydrate. Such a site can be eliminated by substituting another amino acid for Asn or for residue Z, deleting Asn or Z, or inserting a non-Z amino acid between A₁ and Z, or an amino acid other than Asn between Asn and A₁.

Oligonucleotide-directed site-specific mutagenesis procedures can be employed to provide an altered gene having particular codons altered according to the substitution, deletion, or insertion required. Examples of techniques for making such alterations are described in Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462.

IL-4 receptors that may be employed also include derivatives, e.g., various structural forms of the primary protein which retain a desired biological activity. Due to the presence of ionizable amino and carboxyl groups, for example, an IL-4R protein may be in the form of acidic or basic salts, or in neutral form. Individual amino acid residues may also be modified by oxidation or reduction. The primary amino acid structure may be modified by forming covalent or aggregative conjugates with other chemical moieties, such as glycosyl groups, lipids, phosphate, acetyl groups and the like, or by creating amino acid sequence mutants.

PEGylated derivatives (modified with polyethylene glycol) are contemplated. Covalent derivatives may be prepared by linking particular functional groups to IL-4R amino acid side chains or at the N- or C-termini. IL-4R derivatives may also be obtained by cross-linking agents, such as M-maleimidobenzoyl succinimide ester and N-hydroxysuccinimide, at cysteine and lysine residues. IL-4R proteins may also be covalently bound through reactive side groups to various insoluble substrates, such as cyanogen bromide-activated, bisoxirane-activated, carbonyldiimidazole-activated or tosyl-activated agarose structures, or by adsorbing to polyolefin surfaces (with or without glutaraldehyde cross-linking).

IL-4R Fusion Proteins

Other derivatives of IL-4R within the scope of this invention include covalent or aggregative conjugates of IL-4R or its fragments with other proteins or polypeptides, such as by expression of recombinant fusion proteins comprising heterologous polypeptides fused to the N-terminus or C-terminus of an IL-4R polypeptide. The IL-4R portion of the fusion protein can comprise any fragment or derivative as discussed above. The IL-4R fusion proteins can comprise different embodiments including but not limited to fusions of IL-4R to short polypeptides to facilitate purification (e.g., epitope tagging, discussed below), fusion of IL-4R to longer polypeptides to facilitate oligomerization (e.g., Fc domain, discussed below), or fusion to a heterologous polypeptide having a desired biological activity (e.g., inhibiting IL-4 activity, IL-13R discussed below)

For example, the conjugated polypeptide may be a heterologous signal (or leader) peptide, e.g., the yeast alpha-factor leader, or a peptide such as an epitope tag. IL-4R-containing fusion proteins can comprise peptides added to facilitate purification or identification of IL-4R (e.g., poly-His). Specific examples of poly-His fusion constructs that are biologically active are soluble human IL-4R (e.g., comprising residues -2 to 207 or 1-207 of SEQ ID NO:2) -His-His (i.e., soluble human IL-4R fused to two consecutive histidines) and soluble human IL-4R-His-His-His-His-His-His (i.e., soluble human IL-4R fused to six consecutive histidines).

An amino acid sequence of IL-4 receptor can also be linked to the Flag® peptide Asp-Tyr-Lys-Asp-Asp-Asp-Asp-Lys (DYKDDDDK) (SEQ ID NO:3) as described in Hopp et al., Bio/Technology 6:1204, 1988, and U.S. Pat. No. 5,011,912. The Flag® peptide is highly antigenic and provides an epitope reversibly bound by a specific monoclonal antibody, enabling rapid assay and facile purification of expressed recombinant protein. Reagents useful for preparing fusion proteins in which the Flag® peptide is fused to a given polypeptide are commercially available (Sigma, St. Louis, Mo.).

Oligomers that contain IL-4R polypeptides may be employed as IL-4 antagonists. Oligomers may be in the form of covalently-linked or non-covalently-linked dimers, trimers, or higher oligomers. Oligomers comprising two or more IL-4R polypeptides are contemplated for use, with one example being a homodimer. Other oligomers include heterodimers, heterotrimers, and the like, which comprise an IL-4R polypeptide as well as at least one polypeptide that is not derived from the IL-4R of SEQ ID NO:2.

One embodiment is directed to oligomers comprising multiple IL-4R polypeptides joined via covalent or non-covalent interactions between peptide moieties fused to the IL-4R polypeptides. Such peptides may be peptide linkers (spacers), or peptides that have the property of promoting oligomerization. Leucine zippers and certain polypeptides derived from antibodies are among the peptides that can promote oligomerization of IL-4R polypeptides attached thereto, as described in more detail below.

In particular embodiments, the oligomers comprise from two to four IL-4R polypeptides. The IL-4R moieties of the oligomer may be in any of the forms described above, e.g., variants or fragments. Preferably, the oligomers comprise soluble IL-4R polypeptides.

As one alternative, an oligomer is prepared using polypeptides derived from immunoglobulins. Preparation of fusion proteins comprising certain heterologous polypeptides fused to various portions of antibody-derived polypeptides (including the Fc domain) has been described, e.g., by Ashkenazi et al. (PNAS USA 88:10535, 1991); Byrn et al. (Nature 344:677, 1990); and Hollenbaugh and Aruffo (“Construction of Immunoglobulin Fusion Proteins,” in Current Protocols in Immunology, Suppl. 4, pages 10.19.1 - 10.19.11, 1992).

One embodiment of the present invention is directed to a dimer comprising two fusion proteins created by fusing IL-4R to the Fc region of an antibody. A gene fusion encoding the IL-4R/Fc fusion protein is inserted into an appropriate expression vector. IL-4R/Fc fusion proteins are expressed in host cells transformed with the recombinant expression vector, and allowed to assemble much like antibody molecules, whereupon interchain disulfide bonds form between the Fc moieties to yield divalent IL-4R.

The term “Fc polypeptide” as used herein includes native and mutein forms of polypeptides derived from the Fc region of an antibody. Truncated forms of such polypeptides containing the hinge region that promotes dimerization are also included. Fusion proteins comprising Fc moieties (and oligomers formed therefrom) offer the advantage of facile purification by affinity chromatography over Protein A or Protein G columns.

One suitable Fc polypeptide, described in PCT application WO 93/10151 (hereby incorporated by reference), is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al. (EMBO J. 13:3992-4001, 1994). The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

In other embodiments, IL-4R may be substituted for the variable portion of an antibody heavy or light chain. If fusion proteins are made with both heavy and light chains of an antibody, it is possible to form an oligomer with as many as four IL-4R extracellular regions.

Soluble recombinant fusion proteins comprising an IL-4R and various portions of the constant region of an immunoglobulin are described in EP 464,533, along with procedures for preparing such fusion proteins and dimers thereof. Among the fusion proteins described in EP 464,533 are those comprising the extracellular portion of human IL-4R and an Fc polypeptide.

Alternatively, the oligomer is a fusion protein comprising multiple IL-4R polypeptides, with or without peptide linkers (spacer peptides). Among the suitable peptide linkers are those described in U.S. Pat. Nos. 4,751,180 and 4,935,233.

Another method for preparing oligomeric IL-4R involves use of a leucine zipper. Leucine zipper domains are peptides that promote oligomerization of the proteins in which they are found. Leucine zippers were originally identified in several DNA-binding proteins (Landschulz et al., Science 240:1759, 1988), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al. (FEBS Letters 344:191, 1994), hereby incorporated by reference. The use of a modified leucine zipper that allows for stable trimerization of a heterologous protein fused thereto is described in Fanslow et al. (Semin. Immunol 6:267-278, 1994). In one approach, recombinant fusion proteins comprising a soluble IL-4R polypeptide fused to a leucine zipper peptide are expressed in suitable host cells, and the soluble oligomeric IL-4R that forms is recovered from the culture supernatant.

One example of a heterodimer comprises an IL-4R polypeptide derived from the human IL-4R of SEQ ID NO:2, and an IL-2R-gamma polypeptide. IL-2R-gamma (also known as IL-2R-gamma_{c} or -gamma_{c}) is described in U.S. Pat. No. 5,510,259 and in Takeshita et al. (Science 257:379, 17 July 1992), which are incorporated by reference herein. The polypeptides may be in one of the various forms described herein, e.g., soluble fragments, variants, and the like, derived from the indicated proteins. One embodiment of such a heterodimer comprises a soluble IL-4R/Fc fusion protein and a soluble IL-2R-gamma/Fc fusion protein. Such heterodimers are described in WO 96/11213, along with IL-4R homodimers.

Other examples of heterodimers comprise an IL-4R subunit (preferably a soluble fragment of the protein of SEQ ID NO:2) and at least one IL-13 receptor subunit. IL-13 receptor (IL-13R) complexes and IL-13R polypeptides (such as polypeptides designated IL-13R-alpha1 and IL-13R-alpha2) are described in Zurawski et al., J. Biol. Chem. 270 (23), 13869, 1995; de Vries, J. Allergy Clin. Immunol. 102(2):165, August 1998; Callard et al., Immunology Today, 17(3):108, March 1996, and U.S. Pat. No. 5,710,023, each of which is incorporated by reference herein. IL-4R-alpha is reported to be a component of certain multi-subunit IL-13 receptor complexes. One embodiment of a heterodimer provided for use herein comprises a soluble human IL-4R and a soluble IL-13R (preferably a soluble form of the polypeptide described in U.S. Pat. No. 5,710,023 or IL-13R-alpha1). The components of heterodimers may be any suitable form of the polypeptides that retains the desired activity, such as fragments, variants, or fusion proteins (e.g., fusions of soluble receptor polypeptides with Fc polypeptides, leucine zipper peptides, peptide linkers, or epitope tags).

IL-4 receptor polypeptides and fusion proteins described herein may be prepared by any of a number of conventional techniques. IL-4R polypeptides may be purified from cells that naturally express the receptor (such as the cells discussed in Park et al., Proc. Natl. Acad. Sci. USA 84:1669-673, 1987), or may be produced in recombinant expression systems, using well known techniques. Expression systems for use in producing IL-4R include those described in U.S. Pat. No. 5,599,905, which is hereby incorporated by reference.

A variety of expression systems are known for use in producing recombinant proteins. In general, host cells are transformed with a recombinant expression vector that comprises DNA encoding a desired IL-4R polypeptide. Among the host cells that may be employed are prokaryotes, yeast or higher eukaryotic cells. Prokaryotes include gram negative or gram positive organisms, for example E. coli or bacilli. Higher eukaryotic cells include insect cells and established cell lines of mammalian origin. Baculovirus systems for production of heterologous proteins in insect cells are reviewed by Luckow and Summers, Bio/Technology 6:47 (1988). Examples of suitable mammalian host cell lines include the COS-7 line of monkey kidney cells (ATCC CRL 1651) (Gluzman et al., Cell 23:175, 1981), L cells, 293 cells, C127 cells, 3T3 cells (ATCC CCL 163), Chinese hamster ovary (CHO) cells, HeLa cells, BHK (ATCC CRL 10) cell lines, and the CVI/EBNA cell line derived from the African green monkey kidney cell line CVI (ATCC CCL 70) as described by McMahan et al. (EMBO J. 10: 2821, 1991). Appropriate cloning and expression vectors for use with bacterial, fungal, yeast, and mammalian cellular hosts are described by Pouwels et al. (Cloning Vectors: A Laboratory Manual, Elsevier, New York, 1985).

The transformed cells are cultured under conditions that promote expression of the IL-4R, and the polypeptide is recovered by conventional protein purification procedures. One such purification procedure includes the use of affinity chromatography, e.g., over a matrix having IL-4 bound thereto. Expressed IL-4R will be deposited in the cell membrane or secreted into the culture supernatant, depending on the IL-4R DNA selected. Polypeptides contemplated for use herein include substantially homogeneous recombinant mammalian IL-4R polypeptides substantially free of contaminating endogenous materials

Antibodies

Antibodies that function as IL-4 antagonists may be employed in the methods of the present invention. The antibodies preferably are monoclonal antibodies or antigen-binding fragments thereof. Advantageously, humanized or chimeric monoclonal antibodies are employed. Most preferred are human monoclonal antibodies prepared using transgenic mice, as described below. Unless otherwise specified, the term “antibody” as employed herein encompasses all the various forms of antibodies disclosed herein, including but not limited to whole antibodies, antibody fragments, humanized antibodies, human antibodies (e.g., produced in transgenic animals), and immunoglobulin-derived polypeptides produced through genetic engineering techniques, phage display, and the like.

Examples of suitable antibodies are those that interfere with the binding of IL-4 to an IL-4 receptor. Such antibodies, referred to herein as blocking antibodies, may be raised against either IL-4 or IL-4R, and screened in conventional assays for the ability to interfere with binding of IL-4 to IL-4 receptors. Examples of suitable assays are assays that test the antibodies for the ability to inhibit binding of IL-4 to cells expressing IL-4R, or that test antibodies for the ability to reduce a biological or cellular response that results from the binding of IL-4 to cell surface IL-4 receptors. In one embodiment, the antibody is directed against the extracellular domain of the human IL-4R protein of SEQ ID.NO:2.

The human IL-4R protein of SEQ ID NO:2 also is known as IL-4R-alpha. It has been reported that IL-4R-alpha is a component of certain multi-subunit IL-13 receptor complexes (Zurawski et al., J. Biol. Chem. 270 (23), 13869, 1995; de Vries, J. Allergy Clin. Immunol. 102(2):165, August 1998; and Callard et al., Immunology Today, 17(3):108, March 1996, each incorporated by reference herein). Thus, some antibodies raised against IL-4R-alpha may interfere with the binding of IL-13 to such receptor complexes. Such an antibody, when bound to the IL-4R-alpha subunit of such a receptor complex, may physically block or interfere with the binding of IL-13 to the receptor complex. Advantageously, such an antibody binds to the extracellular domain of the IL-4R-alpha protein of SEQ ID NO:2.

In one embodiment, antibodies directed against IL-4R block binding of IL-4 and also IL-13 to cells. The antibodies inhibit IL-4-induced biological activity and also inhibit IL-13-induced activity, and thus may be employed in treating conditions induced by either or both cytokines. Examples of such conditions include but are not limited to oncologic conditions as discussed below, IgE-mediated conditions, asthma, allergic conditions, allergic rhinitis, and dermatitis including atopic dermatitis.

Antibodies that bind to IL-4R and inhibit IL-4 binding may be screened in various conventional assays to identify those antibodies that also interfere with the binding of IL-13 to such receptor complexes. Antibodies may be screened in binding assays or tested for the ability to inhibit an IL-4-induced and an IL-13-induced biological activity. An example of a suitable assay is illustrated in Example 5 below.

Antibodies specific for IL-4 or IL-4R may be prepared by well known procedures. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988).

Antigen-binding fragments of such antibodies may be produced by conventional techniques. Examples of such fragments include, but are not limited to, Fab and F(ab′)₂ fragments. Antibody fragments and derivatives produced by genetic engineering techniques are also contemplated for use.

In one embodiment, an antibody raised against human IL-4R is specific for human IL-4R and does not cross-react with other protein(s). In this particular embodiment, such an antibody lacks cross-reactivity with non-IL-4R proteins, and further lacks cross-reactivity with murine IL-4R or IL-4R derived from other non-human animals, for example.

Additional embodiments include chimeric antibodies, e.g., humanized versions of murine monoclonal antibodies. Such humanized antibodies may be prepared by known techniques, and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment may comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, May, 1993).

A method for producing an antibody comprises immunizing a non-human animal, such as a transgenic mouse, with an IL-4R polypeptide, whereby antibodies directed against the IL-4R polypeptide are generated in said animal. In one alternative, antibodies directed against IL-4 are generated by immunizing a transgenic mouse with an IL-4 polypeptide. Procedures have been developed for generating human antibodies in non-human animals. The antibodies may be partially human, or preferably completely human. For example, transgenic mice into which genetic material encoding one or more human immunoglobulin chains has been introduced may be employed. Such mice may be genetically altered in a variety of ways. The genetic manipulation may result in human immunoglobulin polypeptide chains replacing endogenous immunoglobulin chains in at least some (preferably virtually all) antibodies produced by the animal upon immunization.

Mice in which one or more endogenous immunoglobulin genes have been inactivated by various means have been prepared. Human immunoglobulin genes have been introduced into the mice to replace the inactivated mouse genes. Antibodies produced in the animals incorporate human immunoglobulin polypeptide chains encoded by the human genetic material introduced into the animal.

Examples of techniques for production and use of such transgenic animals are described in U.S. Pat. Nos. 5,814,318, 5,569,825, and 5,545,806, which are incorporated by reference herein. Examples 2-4 below provide further description of the preparation of transgenic mice useful for generating human antibodies directed against an antigen of interest.

Antibodies produced by immunizing transgenic animals with an IL-4R polypeptide are provided herein. Transgenic mice into which genetic material encoding human immunoglobulin polypeptide chain(s) has been introduced are among the suitable transgenic animals. Examples of such mice include, but are not limited to, those containing the genetic alterations described in the examples below. One example of a suitable immunogen is a soluble human IL-4R, such as a polypeptide comprising the extracellular domain of the protein of SEQ ID NO:2, or other immunogenic fragment of the protein of SEQ ID NO:2.

Monoclonal antibodies may be produced by conventional procedures, e.g., by immortalizing spleen cells harvested from the transgenic animal after completion of the immunization schedule. The spleen cells may be fused with myeloma cells to produce hybridomas, by conventional procedures.

A method for producing a hybridoma cell line comprises immunizing such a transgenic animal with an IL-4R immunogen; harvesting spleen cells from the immunized animal; fusing the harvested spleen cells to a myeloma cell line, thereby generating hybridoma cells; and identifying a hybridoma cell line that produces a monoclonal antibody that binds an IL-4R polypeptide. Such hybridoma cell lines, and anti-IL-4R monoclonal antibodies produced therefrom, are encompassed by the present invention. Monoclonal antibodies secreted by the hybridoma cell line are purified by conventional techniques.

Among the uses of such antibodies directed against an IL-4R is use in assays to detect the presence of IL-4R polypeptides, either in vitro or in vivo. The antibodies also may be employed in purifying IL-4R proteins by immunoaffinity chromatography. Those antibodies that additionally can block binding of IL-4 to IL-4R may be used to inhibit a biological activity that results from such binding. Blocking antibodies find use in the methods of the present invention, i.e., treating cancerous conditions as described above. It is also provided that the antibodies can be co-administered with a cytotoxic T-cell agonist.

Such antibodies which function as IL-4 antagonists may also be employed in treating any IL-4-induced condition, including but not limited to asthma and allergies, e.g., allergic rhinitis, contact dermatitis, and atopic dermatitis. In one embodiment, a human anti-IL-4R monoclonal antibody generated by procedures involving immunization of transgenic mice is employed in treating such conditions.

Antibodies may be employed in an in vitro procedure, or administered in vivo to inhibit an IL-4-induced biological activity. Disorders caused or exacerbated (directly or indirectly) by the interaction of IL-4 with cell surface IL-4 receptors thus may be treated. A therapeutic method involves in vivo administration of a blocking antibody to a mammal in an amount effective for reducing an IL-4-induced biological activity.

In one embodiment, human antibodies raised against IL-4R and produced by techniques involving use of transgenic mice, block binding of IL-4 and also IL-13 to cells. Such antibodies are IL-4 antagonists and additionally function as IL-13 antagonists.

Also provided herein are conjugates comprising a detectable (e.g., diagnostic) or therapeutic agent, attached to an antibody directed against IL-4R. Examples of such agents are well known, and include but are not limited to diagnostic radionuclides, therapeutic radionuclides, and cytotoxic drugs. The conjugates find use in in vitro or in vivo procedures.

Further examples of IL-4 antagonists are antibodies that bind IL-4 and inhibit the binding of IL-4 to cell surface receptors. Such antibodies may be prepared, and screened to identify those that are blocking antibodies, by conventional procedures. Antigen-binding fragments of such antibodies find use as antagonists, as do humanized or genetically engineered derivatives thereof.

Examples of procedures for preparing antibodies directed against human IL-4 (including monoclonal antibodies), assays by which blocking antibodies are identified, and techniques for generating humanized or genetically engineered derivatives of anti-IL-4 antibodies, are described in U.S. Pat. Nos. 5,041,381, 5,863,537, 5,928,904, and 5,676,940, which are hereby incorporated by reference. Further examples of antibodies that may be employed as IL-4 antagonists are described in WO 91/09059, also incorporated by reference herein.

Other antagonists

Any compound that functions as an IL-4 antagonist and is suitable for administration in accordance with the methods of the present invention may be employed. Antagonists need not completely abolish IL-4-induced biological activity to be useful. Rather, a given antagonist may reduce a biological activity of IL-4.

Derivatives, mutants/muteins, and other variants of IL-4 that function as IL-4 antagonists may be employed. Peptides (which may or may not be muteins) derived from IL-4 that bind to an IL-4R without inducing transduction of a biological signal find use herein. Such peptides function as inert blockers, interfering with the binding of biologically active endogenous IL-4 to cell surface receptors. IL-4-induced signal transduction thereby is inhibited. Muteins or other antagonists that induce a biological response at a reduced level or to a lesser degree, compared to the response induced by native IL-4, also find use as IL-4 antagonists.

Further examples of IL-4 antagonists, including IL-4 muteins, and procedures for preparation thereof are described in Muller et al., J. Mol. Biol., 237:423-436, 1994; U.S. Pat. No. 6,028,176, and U.S. Pat. No. 5,723,118, which are each incorporated by reference herein.

Other options are antisense molecules (oligonucleotides) that inhibit expression of IL-4. Alternatively, the antisense molecule may suppress expression of other molecules involved in IL-4-induced signal transduction.

Any suitable assay, including in vitro assays, can be utilized to determine whether a given compound inhibits an IL-4-induced biological activity. An IL-4 antagonist may be assayed for the ability to inhibit ³H-thymidine incorporation in cells that normally proliferate in response to IL-4. Such an assay may be employed in testing an IL-4 antagonist for the ability to inhibit ³H-thymidine incorporation in a particular type of cancer cells.

An alternative involves use of conventional binding assay techniques to test an antagonist for the ability to inhibit binding of IL-4 to cells expressing native or recombinant IL-4 receptors. For use in such assays, recombinant human IL-4 can be expressed and purified as described in U.S. Pat. No. 5,017,691, hereby incorporated by reference herein, or in Park et al., J. Exp. Med. 166476 (1987). The purified protein may be labeled with a detectable agent (e.g., radiolabeled) by any of a number of conventional techniques. A commercially available enzymobead radioiodination reagent (BioRad) may be employed in radiolabeling IL-4 with ¹²⁵I for example.

It is contemplated that any IL-4 inhibitor can be used in the methods of the invention. In addition any IL-4 inhibitor may be co-administered with a cytotoxic T-cell agonist, e.g., interferon-alpha.

Co-administration of Interferon with IL-4 antagonists

Interferons are a group of proteins naturally produced in the body in response to an exterior stimulus, for example virus causing an infection. Interferon may also be present in small quantities in the blood in connection with certain virus and tumor diseases. The three main types of interferons are: interferon-alpha produced by leukocytes; beta-interferon produced by binding tissue cells; and gamma-interferon produced by immuno-competent cells. Native interferon produced from leukocytes consists of a native mixture of different interferon-alpha subtypes or interferon components.

The different interferon-alpha components are named Interferon-alpha-1, Interferon-alpha-2, Interferon-alpha-3, etc. Moreover, there is allelic variation within the different alpha-interferon components termed Interferon-alpha-2a, -alpha-2b, -alpha-2c, etc. Human interferon-alpha comprises a family having at least 14 different types encoded by at least nine different genes, all of which are located on chromosome nine. The interferon-alpha family members have relatively high homology of 75-85% to each other, and typically are 165-166 amino acids in length, including a 20 amino acid signal peptide. See U.S. Pat. No. 5,391,713 for general discussion and background on interferons, and also purification methods of interferons.

Some indications for which recombinant interferons have been approved as a therapeutic include: hairy cell leukemia; chronic myelogenous leukemia; myeloma; lymphomas; melanomas; and renal cancer among others. Within the interferon-alpha family, interferon-alpha2 is the most widely approved family member for therapeutic uses (see Biron, 2001, Immunity, 14:661-664; Gutterman, 1994, Proc. Nat'l. Acad. Sci., 91:1198-1205; and Borden et al., 2000, Cancer Biology, 10:125-144).

In one embodiment of the present invention, interferon-alpha is used as a cytotoxic T-cell immune stimulant for cancer therapy in conjunction with an antagonist of IL-4. In a particular embodiment, an effective amount of interferon-alpha2 is co-administered with an effective amount of an antagonist of IL-4 to a cancer patient in need thereof. Examples of cancers that can be treated in this embodiment include, but are not limited to renal cancer, Burkitt lymphoma, Hodgkin's lymphoma, non-Hodgkin's lymphoma and Sezary syndrome. In another embodiment, the patient in need of treatment for cancer has a T-cell immune phenotype of predominantly TH2-type cells.

In one embodiment, a cancer patient who fails to respond to interferon-alpha treatment, as measured by failure to slow or terminate tumor progression or failure to induce secondary cytokines in response to interferon-alpha, has interferon-alpha responsiveness restored by co-administration of interferon-alpha with an antagonist of IL-4. In another embodiment, a cancer patient who responds to interferon-alpha treatment has interferon-alpha responsiveness enhanced by co-administration of interferon-alpha with an antagonist of IL-4.

Restoration or enhancement of a cancer patient's responsiveness to interferon-alpha treatment can be measured using any standard cell-based or biochemical assay well known in the art. For example, expression of secondary cytokines in response to interferon-alpha can be measured, e.g., by an ELISA, as an indirect measure of the effectiveness of the treatment. Likewise, interferon-alpha stimulation of a cytotoxic T-cell response in a patient can be measured by a reduced or steady-state tumor burden in the patient subsequent to treatment.

In yet another embodiment, the invention contemplates that additional therapeutics can be delivered to a cancer patient upon co-administration of an IL-4 antagonist with interferon-alpha. For example, IL-12 has been shown to enhance interferon-alpha antitumor response against colon and renal cancer cells (Mendiratta et al., 2000, Human Gene Ther., 11:1851-1862). As such, any anti-cancer chemotherapy agent, including but not limited to cisplatin, carboplatin, taxol, etoposide, Novantrone® (mitoxantrone), vinblastine, vincristine, 5-fluorouracil, actinomycin D, camptothecin (or water soluble derivatives thereof), methotrexate, mitomycin (e.g., mitomycin C), cyclohexamide, gemcitabine, dacarbazine (DTIC), procarbazine, cytosine arabinoside (Ara-C), cyclophosphamide, thiotepa, cytoxin, leucovorin, melphalan, busulfan, and anti-neoplastic antibiotics such as adriamycin (doxorubicin), daunomycin, and bleomycin, or any biological chemotherapy agent such as, but not limited to IL-12, can precede, follow or coincide with the treatments according to the methods described herein.

PEGylated derivatives of interferon-alpha (modified with polyethylene glycol) are also contemplated for use in the methods of the present invention. In one embodiment, interferon-alpha is prepared such that the protein compositions are linked to PEG to prolong plasma life. (Bukowski et al., 1999, Proc Am Soc Clin Oncol 18:446a).

Covalent derivatives of interferon-alpha may be prepared by linking particular functional groups to interferon-alpha amino acid side chains or at the N- or C-termini. Interferon-alpha derivatives may also be obtained by cross-linking agents, such as M-maleimidobenzoyl succinimide ester and N-hydroxysuccinimide, at cysteine and lysine residues. Interferon-alpha proteins may also be covalently bound through reactive side groups to various insoluble substrates, such as cyanogen bromide-activated, bisoxirane-activated, carbonyldiimidazole-activated or tosyl-activated agarose structures, or by adsorbing to polyolefin surfaces (with or without glutaraldehyde cross-linking).

Therapeutic Methods and Administration of Antagonists and Agonists

Methods provided herein comprise administering an IL-4 antagonist to a patient in vivo, thereby reducing an IL-4-induced biological response that plays a role in a particular form of cancer or a cancer-associated condition. Additional methods provided herein comprise co-administering to a cancer patient in vivo, an IL-4 antagonist and a cytotoxic T-cell agonist such as a cytokine, e.g., interferon-alpha, thereby treating the cancer or cancer-associated condition. Other methods of the invention involve contacting endogenous IL-4 with an IL-4 antagonist in an ex vivo procedure.

Treatment encompasses alleviation of at least one symptom of the patient's disease, or reduction of disease severity, and the like. An IL-4 antagonist need not effect a complete “cure”, or eradicate every symptom or manifestation associated with oncologic disease, to constitute a viable therapeutic agent. As is recognized in the pertinent field, drugs employed as therapeutic agents may reduce the severity of a given disease state, but need not abolish every manifestation of the disease to be regarded as useful therapeutic agents.

An IL-4 antagonist may be administered in an amount effective in reducing proliferation of cancer cells. An IL-4 antagonist may be administered in an amount sufficient to prolong patient survival. In particular embodiments, an IL-4 antagonist is administered in an amount sufficient to reduce tumor burden, tumor size or the number of tumor sites in a patient. The tumors may be primary or metastatic. One embodiment of the invention is directed to a method comprising administering to a patient an IL-4 antagonist in an amount and for a time sufficient to induce a sustained improvement over baseline of an indicator that reflects the severity of a particular oncologic disorder.

An IL-4 antagonist may also be co-administered with a cytotoxic T-cell agonist in effective amounts to reduce proliferation of cancer cells and to stimulate an immune response to the cancer. The IL-4 antagonist and cytotoxic T-cell agonist can be co-administered in an amount sufficient to prolong patient survival. In particular embodiments, an IL-4 antagonist and cytotoxic T-cell agonist are administered in amounts sufficient to reduce the cancer burden, i.e., to reduce the cancer cell burden, to reduce the tumor burden, tumor size or the number of tumor sites in a patient. One embodiment of the invention is directed to a method comprising co-administering to a patient an IL-4 antagonist and cytotoxic T-cell agonist in amounts and for a time sufficient to induce a sustained improvement over baseline of an indicator that reflects the severity of a particular oncologic disorder.

To monitor the effectiveness of an IL-4 antagonist, or an IL-4 antagonist co-administered with an agonist of cytotoxic T-cells, a patient's tumor burden may be assessed by any of a number of conventional techniques. Suitable procedures vary according to the type of cancer, but include various tumor imaging techniques, or procedures for determining the amount of a given tumor-associated antigen or protein in a patient's blood or urine, for example.

As is understood in the pertinent field, antagonists are administered to a patient in a manner appropriate to the indication. Likewise, the therapeutics of the invention, for example an IL-4 antagonist or an IL-4 antagonist that is co-administered with a cytotoxic T-cell agonist, e.g., interferon-alpha, are given in a manner appropriate for the indication. One factor to be considered is the location(s) of a patient's tumor(s), including any metastatic sites.

Antagonists (with or without interferon-alpha) may be administered by any suitable technique, including but not limited to parenterally, topically, or by inhalation. Localized administration at a site of disease is contemplated, as are transdermal delivery and sustained release from implants. If injected, the antagonist (with or without interferon-alpha) can be administered, for example, via intra-articular, intravenous, intramuscular, intratumoral, intralesional, intraperitoneal or subcutaneous routes, by bolus injection, or continuous infusion. Other alternatives include eyedrops; oral preparations including pills, syrups, lozenges or chewing gum; and topical preparations such as lotions, gels, sprays, and ointments. For treating lung cancer, or pulmonary metastases of other forms of cancer, the IL-4 antagonist preferably is delivered by inhalation. Delivery by inhalation includes, for example, nasal or oral inhalation, use of a nebulizer, inhalation of the antagonist in aerosol form, and the like.

Use of IL-4 antagonists in ex vivo procedures is contemplated. For example, a patient's blood (bodily fluid containing IL-4) may be contacted with an antagonist that binds IL-4 ex vivo, thereby reducing the amount of IL-4 in the fluid when returned to the patient.

Use of IL-4 antagonists with interferon-alpha in ex vivo procedures is also contemplated. For example, a patient's blood (bodily fluid containing IL-4) may be contacted with an antagonist that binds IL-4 ex vivo, thereby reducing the amount of IL-4 and enhancing the activation of the cytotoxic T-cells in the fluid when returned to the patient. The antagonist (with or without interferon-alpha) may be bound to a suitable insoluble matrix or solid support material.

Advantageously, antagonist (with or without interferon-alpha) are administered in the form of a composition comprising at least one IL-4 antagonist and one or more additional components such as a physiologically acceptable carrier, excipient or diluent. The present invention provides such compositions comprising an effective amount of an IL-4 antagonist, for use in the methods provided herein.

The compositions contain antagonist(s) (with or without interferon-alpha) in any of the forms described herein. The antagonist may be a whole antibody or an antigen-binding fragment or engineered derivative thereof, for example. For compositions containing an IL-4 receptor, the receptor may be any of the fragments, variants, or oligomers of the protein of SEQ ID NO:2 described herein, for example.

Compositions may, for example, comprise an IL-4 antagonist together with a buffer, antioxidant such as ascorbic acid, low molecular weight polypeptide (such as those having fewer than 10 amino acids), protein, amino acid, carbohydrate such as glucose, sucrose or dextrins, chelating agents such as EDTA, glutathione, and other stabilizers and excipients. Neutral buffered saline or saline mixed with conspecific serum albumin are examples of appropriate diluents. In accordance with appropriate industry standards, preservatives such as benzyl alcohol may also be added. The composition may be formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in Remington's Pharmaceutical Sciences, 16^{th} Ed., Mack Publishing Company, Easton, Pa., 1980.

Kits for use by medical practitioners include an IL-4 antagonist (with or without interferon-alpha) and a label or other instructions for use in treating any of the conditions discussed herein. The kit preferably includes a sterile preparation of one or more IL-4 antagonists, which may be in the form of a composition as disclosed above, and may be in one or more vials.

Dosages and the frequency of administration may vary according to such factors as the route of administration, the particular antagonist employed, the nature and severity of the disease to be treated, whether the condition is acute or chronic, and the size and general condition of the patient. Appropriate dosages can be determined by procedures known in the pertinent art, e.g. in clinical trials that may involve dose escalation studies.

In one embodiment, antagonist(s) are administered after a patient is diagnosed with cancer. An IL-4 antagonist (with or without interferon-alpha) may be administered once, or repeatedly. In particular embodiments, the antagonist is administered over a period of at least a month or more, e.g., for one, two, or three months or even indefinitely. Depending upon such factors as the type and size of a patient's tumors, administration for from one to six weeks may be sufficient. Where the treatment comprises co-administration of an IL-4 antagonist of the invention and interferon-alpha, the antagonist can be administered once, or repeatedly either together or followed by administration of interferon-alpha. In general, the IL-4 antagonist (with or without interferon-alpha) is administered until the patient manifests a medically relevant degree of improvement over baseline for the chosen indicator or indicators.

As one option, after a therapeutic goal is achieved, such as reduction or elimination of tumors, treatment may continue to suppress further tumor formation or growth. Maintenance doses may be administered.

Further alternatives involve administering an IL-4 antagonist to a patient who is at risk for cancer, but has not been diagnosed with cancer, or who has a precancerous condition, e.g., in whom cells characterized as precancerous have been detected. IL-4 antagonists find use in preventing development or differentiation of cancer cells.

In one embodiment, an IL-4 inhibitor is administered to a patient who has a solid tumor, in an amount and for a period of time sufficient to inhibit angiogenesis (neo-vascularization) of the tumor. In this embodiment, the IL-4 inhibitor preferably is administered subcutaneously.

Another embodiment is directed to a method comprising administering an IL-4 antagonist to a cancer patient, in an amount and for a time sufficient to promote a TH1-type immune response in the patient. The IL-4 antagonist serves to increase the proportion of helper T-cells in the patient that secrete TH1-type cytokines. Yet another embodiment is directed to co-administering an IL-4 antagonist to a cancer patient with interferon-alpha, in amounts and times sufficient to promote a TH1-type immune response in the patient. The IL-4 antagonist increases the proportion of helper T-cells in the patient that secrete TH1-type cytokines thereby increasing the number of activated cytotoxic T-cells, which can then be further activated by interferon-alpha. In non-limiting examples of such a method(s), the patient is afflicted with a bronchogenic carcinoma or renal carcinoma, or is receiving an anti-cancer vaccine or undergoing immunotherapy.

An additional embodiment is directed to a method comprising administering an IL-4 antagonist to a cancer patient, in an amount and for a time sufficient to increase apoptosis of cancer cells in the patient. In an additional embodiment, an IL-4 antagonist is co-administered to a cancer patient with interferon-alpha in an amount sufficient to increase apoptosis of cancer cells in the patient. In one example of such a method, the patient is afflicted with B-CLL. In another example of such a method, the patient is afflicted with Sezary syndrome.

Particular embodiments of the present invention involve administering an IL-4 antagonist at a dosage of from about 1 ng/kg/day to about 10 mg/kg/day, more preferably from about 500 ng/kg/day to about 5 mg/kg/day, and most preferably from about 5 ug/kg/day to about 2 mg/kg/day, to a patient. In additional embodiments, an IL-4 antagonist such as a soluble human IL-4R polypeptide is administered to adults one time per week, two times per week, or three or more times per week, to treat the medical disorders disclosed herein. If injected, the effective amount of antagonist per adult dose may range from 1-20 mg/m², and preferably is about 5-12 mg/m². Alternatively, a flat dose may be administered; the amount may range from 5-100 mg/dose. One range for a flat dose is about 20-30 mg per dose. In one embodiment of the invention, a flat dose of 25 mg/dose is repeatedly administered by injection. If a route of administration other than injection is used, the dose is appropriately adjusted in accordance with standard medical practices. One example of a therapeutic regimen involves injecting a dose of about 20-30 mg of IL-4R or other antagonist one to three times per week over a period of at least three weeks, though treatment for longer periods may be necessary to induce the desired degree of improvement. For pediatric patients (age 4-17), one suitable regimen involves the subcutaneous injection of 0.4 mg/kg, up to a maximum dose of 25 mg of IL-4R, administered two or three times per week.

Particular embodiments of the methods provided herein involves subcutaneous injection of from 0.5 mg to 10 mg, preferably from 3 to 5 mg, of a soluble IL-4R, once or twice per week. Another embodiment is directed to pulmonary administration (e.g., by nebulizer) of 3 or more mg of a soluble IL-4R once a week.

Another example of a therapeutic regimen provided herein comprises administering a soluble human IL-4R to a human who has Burkitt lymphoma, at a dosage of from 1.5 to 3.0 mg, administered once a week. The IL-4R may be administered by subcutaneous injection.

An IL-4 antagonist is administered to the patient in an amount and for a time sufficient to induce an improvement, preferably a sustained improvement, in at least one indicator that reflects the severity of the disorder that is being treated. Various indicators that reflect the extent of the patient's illness may be assessed for determining whether the amount and time of the treatment is sufficient. Such indicators include, for example, clinically recognized indicators of disease severity, symptoms, or manifestations of the disorder in question. In most instances, an improvement is considered to be sustained if the patient exhibits the improvement on at least two occasions separated by two to four weeks. The degree of improvement generally is determined by the patient's physician, who may make this determination based on signs or symptoms, and who may also employ questionnaires that are administered to the patient, such as quality-of-life questionnaires developed for a given disease.

Particular embodiments of methods and compositions of the invention involve the use of two or more different IL-4 antagonists. In further embodiments, IL-4 antagonist(s) are administered alone or in combination with other agents useful for treating the condition with which the patient is afflicted. Examples of such agents include both proteinaceous and non-proteinaceous drugs. When multiple therapeutics are co-administered, dosages may be adjusted accordingly, as is recognized in the pertinent art. “Co-administration” and combination therapy are not limited to simultaneous administration, but include treatment regimens in which an IL-4 antagonist is administered at least once during a course of treatment that involves administering at least one other therapeutic agent to the patient.

Examples of other agents that may be co-administered with IL-4 antagonists are other antibodies, cytokines, or cytokine receptors, which are chosen according to the particular condition to be treated. Alternatively, non-proteinaceous drugs that are useful in treating one of the particular conditions discussed above may be co-administered with an IL-4 antagonist.

IL-4 antagonists may be co-administered with one or more other anti-cancer agent(s). Examples of such agents include both proteinaceous and non-proteinaceous drugs, and radiation therapy. Among the texts providing guidance for cancer therapy is Cancer, Principles and Practice of Oncology, 4th Edition, DeVita et al., Eds. J. B. Lippincott Co., Philadelphia, Pa. (1993). Drugs employed in cancer treatment may have a cytotoxic or cytostatic effect on cancer cells, or may reduce proliferation of the malignant cells. An appropriate therapeutic approach is chosen according to such factors as the particular type of cancer and the general condition of the patient, as is recognized in the pertinent field.

A wide variety of drugs have been employed in chemotherapy of cancer. Examples include, but are not limited to, cisplatin, carboplatin, taxol, etoposide, Novantrone® (mitoxantrone), vinblastine, vincristine, 5-fluorouracil, actinomycin D, camptothecin (or water soluble derivatives thereof), methotrexate, mitomycin (e.g., mitomycin C), cyclohexamide, gemcitabine, dacarbazine (DTIC), procarbazine, cytosine arabinoside (Ara-C), cyclophosphamide, thiotepa, cytoxin, leucovorin, melphalan, busulfan, and anti-neoplastic antibiotics such as adriamycin (doxorubicin), daunomycin, and bleomycin.

For in vivo use, derivatives of camptothecene that are more water soluble are advantageously employed. Examples of such water soluble derivatives are the drugs 7-ethyl-10-[4-(1-piperidino)-1-piperidino]carbonyloxy-camptothecin (CPT-11; irinotecan) and 9-dimethyl-aminomethyl-10-hydroxycamptothecin (topotecan). Camptothecene and the two above-described derivatives are DNA topoisomerase 1 inhibitors.

In particular embodiments, a cytotoxic agent is attached to a targeting moiety that delivers the agent to cancer cells. Examples of such targeting moieties include but are not limited to, antibodies that bind to cancer cells. Another example of a targeting moiety is CD30 ligand (CD30-L), which binds to CD30⁺ cancer cells such as Hodgkin's Disease (Reed/Sternberg) cells and a number of CD30⁺ non-Hodgkin's lymphoma cells. The cytotoxic agent may be a radionuclide, chemotherapy drug, or protein toxin, for example. CD30-L/cytotoxic agent conjugates are further described in U.S. Pat. No. 5,753,203, hereby incorporated by reference.

In particular embodiments, an IL-4 antagonist may be co-administered with another protein in cancer therapy. Examples include various cytokines or cytokine receptors. The co-administered protein may directly or indirectly kill cancer cells, or contribute to a desired immune or other biological response that helps combat cancer. One such protein is TNF-Related Apoptosis-Inducing Ligand (TRAIL, described in U.S. Pat. No. 5,763,223, hereby incorporated by reference), which induces apoptosis of a number of cancer cell types. Other proteins that may be co-administered include antibodies useful in cancer therapy, with one example being antibodies directed against the Her-2 antigen. Examples of anti-Her-2 antibodies, including monoclonal antibodies and fragments thereof, and the ability of the antibodies to inhibit proliferation or induce apoptosis of various types of tumor cells, e.g., several carcinoma cell types, are discussed in WO 99/48527, hereby incorporated by reference. Such antibodies may be co-administered to treat cancers that include but are not limited to carcinomas, such as breast carcinoma. One embodiment comprises co-administering an IL-4 inhibitor and the anti-Her-2 antibody-based drug Herceptin to a cancer patient.

Another example comprises co-administering an IL-4 inhibitor with a tumor necrosis factor (TNF) inhibitor to a cancer patient. In one particular embodiment, the cancer patient has Burkitt Lymphoma. One example involves co-administering an IL-4 antagonist and a TNF antagonist to a Burkitt Lymphoma patient. TNF antagonists that inhibit TNF-alpha and/or TNF-beta are known, including antibodies and TNF receptor (TNF-R) polypeptides, for example, with the latter including the TNF-R and fusions thereof described in WO 91/03553 and U.S. Pat. No. 5,395,760, hereby incorporated by reference. A preferred TNF antagonist is a dimer comprising two soluble TNF-R/Fc fusion proteins (comprising an extracellular domain of a human TNF-R and an Fc region polypeptide derived from an antibody), such as etanercept (Enbrel®; Immunex Corporation), which may be administered by subcutaneous injection or other suitable means.

To further promote a TH1-type immune response, one therapeutic approach comprises co-administration of an antagonist of IL-4 with an antagonist of at least one other cytokine associated with a TH2 immune response. For example, an IL-4 antagonist may be co-administered with antagonist(s) of one or more additional TH2-type cytokines, including but not limited to co-administration of IL-4 antagonist(s) with IL-5 antagonist(s) and/or IL-10 antagonist(s). CD30 ligand (CD30-L) has been reported to promote a TH2 response; thus, an antagonist of CD30-L may be co-administered with an IL-4 antagonist.

To further promote a TH1-type immune response, one therapeutic approach comprises co-administering an antagonist of IL-4 and an agonist of TH1-cell immune response activation. For example, an IL-4 antagonist may be co-administered with one or more agonists of TH1-type immune response, including but not limited to co-administration of IL-4 antagonist(s) with interferon-alpha2.

IL-5 antagonists include but are not limited to molecules that interfere with the binding of IL-5 to an IL-5 receptor, such as an anti-IL-5 antibody, an antibody that binds an IL-5 receptor, or a receptor such as a soluble human IL-5 receptor polypeptide. Likewise, examples of IL-10 antagonists are molecules that interfere with the binding of IL-10 to an IL-10 receptor, such as an anti-IL-10 antibody, an anti-IL-10 receptor antibody, or a receptor such as a soluble human IL-10 receptor polypeptide. CD30-L antagonists include CD30 (especially soluble CD30), antibodies that bind CD30, and antibodies that bind CD30-L. Suitable antibodies are blocking antibodies, in that they inhibit the binding of the cytokine to a cell surface receptor. The antibodies preferably are monoclonal antibodies, and may be whole antibodies, antigen-binding fragments of the antibodies, human or humanized monoclonal antibodies, for example.

In one therapeutic approach, an IL-4 antagonist is co-administered with one or more additional agents that inhibit angiogenesis, to treat cancer. Inhibition of angiogenesis may be confirmed by known procedures, such as microvessel density counts.

As discussed above, administration of IL-4 antagonists inhibits the apoptosis-suppressing activity of IL-4, thereby allowing apoptosis of cancer cells to occur. Other agents that promote apoptosis of cancer cells may be co-administered with the IL-4 antagonist. One example is an inhibitor of BcI-2, which is an apoptosis suppressor. Antisense molecules that inhibit BcI-2 expression may be employed, for example. Another example is TRAIL, described above.

Another embodiment of the present invention is directed to a method for treating cancer, comprising administering an IL-13 antagonist to a cancer patient. IL-13 antagonists may be employed in treating any type of cancer that is caused or exacerbated, directly or indirectly, by IL-13, such as any form of cancer in which IL-13 promotes growth or proliferation of the cancer cells or otherwise plays a role in pathogenesis of the disease. Conventional proliferation assays may be employed in identifying types of cancer cells that proliferate in response to IL-13. IL-13 antagonists find use when IL-13 functions as a growth factor, e.g., an autocrine growth factor, for a type of cancer cells in the patient's tumor, for example.

One type of cancer for which IL-13 antagonists may be administered is Hodgkin's Disease, characterized by Reed-Sternberg cells (Hodgkin/Reed-Sternberg cells) within the tumor. Further examples are certain non-Hodgkin's lymphomas (NHL), including but not limited to follicular small cell NHL, diffuse small cell NHL, diffuse large cell NHL, and poppema lymphoma.

Any suitable antagonist that inhibits biological activity of IL-13 may be employed. Examples of IL-13 antagonists include but are not limited to IL-13 receptors (preferably soluble forms thereof), IL-13 receptor antagonists, antibodies directed against IL-13, antibodies directed against an IL-13 receptor, other proteins that interfere with the binding of IL-13 to an IL-13 receptor, and compounds that inhibit IL-13-mediated signal transduction. IL-13 receptors and heterodimers comprising IL-13R polypeptides as components thereof are described above.

Some antibodies raised against IL-4 receptor immunogens function as IL-13 antagonists, as discussed above. The human IL-4R-alpha of SEQ ID NO:2, preferably the extracellular domain thereof, may be employed as an immunogen in producing antibodies, which then may be screened to identify those capable of functioning as IL-13 antagonists. Particular methods of the present invention comprise administering such an anti-IL-4R antibody, which functions as an IL-13 antagonist, to a patient who has Hodgkin's Disease or non-Hodgkin's lymphoma.

IL-13 antagonists may be formulated in pharmaceutical compositions, and administered by various routes, as described above in connection with IL-4 antagonists. IL-13 antagonists may be co-administered with other therapeutic agents. Examples of combination therapy comprise co-administration of an IL-13 antagonist with one of more of the following to a Hodgkin's Disease (HD) or non-Hodgkin's lymphoma (NHL) patient: a chemotherapeutic drug; CD30 Ligand; a conjugate comprising a cytotoxic agent attached to a CD30 ligand polypeptide; and a conjugate comprising a cytotoxic agent attached to an antibody that binds CD30. Use of such conjugates for delivering cytotoxic agents to CD30⁺ HD or NHL patients is discussed above and in U.S. Pat. No. 5,753,203.

Also provided herein is a method for suppressing IL-4-induced and IL-13-induced activities in a human, comprising administering to the human an effective amount of an antibody that inhibits binding of both IL-4 and IL-13 to cells. Conditions induced by IL-4 or by IL-13, or by both cytokines, thus may be treated. Such conditions include, for example, any type of cancer in which IL-4 and IL-13 both play a role, e.g., cancer cell types for which IL-4 and IL-13 are growth factors in that IL-4 and IL-13 each promote growth of the cancer cells. Examples include non-Hodgkin's lymphomas, including but not limited to those listed above, as can be determined in conventional cell proliferation assays.

A method for treating cancer comprises administering antagonist(s) that inhibit biological activity of IL-4 and IL-13. The IL-4 antagonist and IL-13 antagonist may be different compounds. Alternatively, a single compound may function as an antagonist of both IL-4 and IL-13, with one example being the above-described antibodies raised against IL-4R that also inhibit binding of IL-13 to receptors.

An IL-4 antagonist and interferon-alpha are co-administered to the patient in amounts and for a time sufficient to induce an improvement, preferably a sustained improvement, in at least one indicator that reflects the severity of the disorder that is being treated. Various indicators that reflect the extent of the patient's illness may be assessed for determining whether the amount and time of the treatment is sufficient. Such indicators include, for example, clinically recognized indicators of disease severity, symptoms, or manifestations of the disorder in question. In most instances, an improvement is considered to be sustained if the patient exhibits the improvement on at least two occasions separated by two to four weeks. The degree of improvement generally is determined by the patient's physician, who may make this determination based on signs or symptoms, and who may also employ questionnaires that are administered to the patient, such as quality-of-life questionnaires developed for a given disease.

The following examples are offered by way of illustration, and not by way of limitation.

### EXAMPLE 1

### Preparation of Monoclonal Antibodies

IL-4 receptor polypeptides may be employed as immunogens in generating monoclonal antibodies by conventional techniques, e.g., techniques described in U.S. Pat. No. 5,599,905, hereby incorporated by reference. It is recognized that polypeptides in various forms may be employed as immunogens, e.g., full length proteins, fragments thereof, fusion proteins thereof such as Fc fusions, cells expressing the recombinant protein on the cell surface, etc.

To summarize an example of such a procedure, an IL-4R immunogen emulsified in complete Freund's adjuvant is injected subcutaneously into Lewis rats, in amounts ranging from 10-100 μl. Three weeks later, the immunized animals are boosted with additional immunogen emulsified in incomplete Freund's adjuvant and boosted every three weeks thereafter. Serum samples are periodically taken by retro-orbital bleeding or tail-tip excision for testing by dot-blot assay, ELISA (enzyme-linked immunosorbent assay), or inhibition of binding of ¹²⁵I-IL-4 to extracts of IL-4R-expressing cells. Following detection of an appropriate antibody titer, positive animals were given a final intravenous injection of antigen in saline. Three to four days later, the animals are sacrificed, splenocytes harvested, and fused to the murine myeloma cell line AG8653. The resulting hybridoma cell lines are plated in multiple microtiter plates in a HAT selective medium (hypoxanthine, aminopterin, and thymidine) to inhibit proliferation of non-fused cells, myeloma hybrids, and spleen cell hybrids.

Hybridoma clones thus generated are screened for reactivity with IL-4R. Initial screening of hybridoma supernatants utilizes an antibody capture and binding of partially purified ¹²⁵I-IL-4 receptor. Hybridomas that are positive in this screening method are tested by a modified antibody capture to detect hybridoma cells lines that are producing blocking antibody. Hybridomas that secrete a monoclonal antibody capable of inhibiting ¹²⁵I-IL-4 binding to cells expressing IL-4R are thus detected. Such hybridomas then are injected into the peritoneal cavities of nude mice to produce ascites containing high concentrations (>1 mg/ml) of anti-IL-4R monoclonal antibody. The resulting monoclonal antibodies may be purified by ammonium sulfate precipitation followed by gel exclusion chromatography, and/or affinity chromatography based on binding of antibody to Protein G.

### EXAMPLE 2

### Generation of Cmu Targeted Mice

This example describes procedures for generating transgenic mice. Additional procedures for generating transgenic mice, and the use of such mice for preparing human antibodies, are described in Examples 3 and 4.

Construction of a CMD targeting vector. The plasmid pICEmu contains an EcoRI/XhoI fragment of the murine Ig heavy chain locus, spanning the mu gene, that was obtained from a Balb/C genomic lambda phage library (Marcu et al., Cell 22: 187, 1980). This genomic fragment was subcloned into the XhoI/EcoRI sites of the plasmid pICEMI9H (Marsh et al; Gene 32, 481-485, 1984). The heavy chain sequences included in pICEmu extend downstream of the EcoRI site located just 3′ of the mu intronic enhancer, to the XhoI site located approximately 1 kb downstream of the last transmembrane exon of the mu gene; however, much of the mu switch repeat region has been deleted by passage in E. coli.

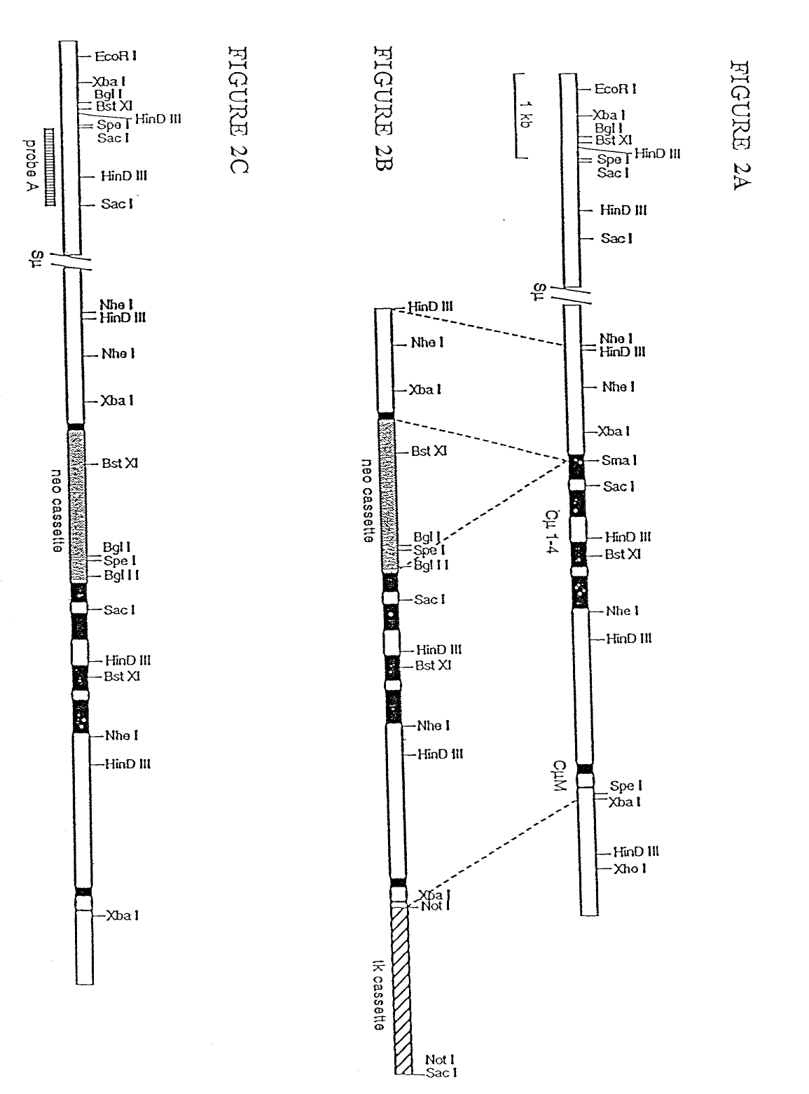

The targeting vector was constructed as follows. (See FIGS. 2A-2C, which depict further details.) A 1.3 kb HindIII/SmaI fragment was excised from pICEmu and subcloned into HindIII/SmaI digested pBluescript (Stratagene, La Jolla, Calif.). This pICEmu fragment extends from the HindIII site located approximately 1 kb 5′ of Cmu1 to the SmaI site located within Cmu1. The resulting plasmid was digested with SmaI/SpeI and the approximately 4 kb SmaI/XbaI fragment from pICEmu, extending from the SmaI site in Cmu1 3′ to the XbaI site located just downstream of the last Cmu exon, was inserted. The resulting plasmid, pTAR1, was linearized at the SmaI site, and a neo expression cassette inserted. This cassette consists of the neo gene under the transcriptional control of the mouse phosphoglycerate kinase (pgk) promoter (XbaI/TaqI fragment; Adra et al. (1987) Gene 60: 65-74) and containing the pgk polyadenylation site (PvuII/HindIII fragment; Boer et al. (1990) Biochemical Genetics 28: 299-308). This cassette was obtained from the plasmid pKJ1 (described by Tybulewicz et al. (1991) Cell 65: 1153-1163) from which the neo cassette was excised as an EcoRI/HindIII fragment and subcloned into EcoRI/HindIII digested pGEM-7Zf (+) to generate pGEM-7 (KJ1). The neo cassette was excised from pGEM-7 (KJ1) by EcoRI/SaII digestion, blunt ended and subcloned into the SmaI site of the plasmid pTAR1, in the opposite orientation of the genomic Cmu sequences.

The resulting plasmid was linearized with Not I, and a herpes simplex virus thymidine kinase (tk) cassette was inserted to allow for enrichment of ES clones bearing homologous recombinants, as described by Mansour et al. (1988) Nature 336: 348-352. This cassette consists of the coding sequences of the tk gene bracketed by the mouse pgk promoter and polyadenylation site, as described by Tybulewicz et al. (1991) Cell 65:1153-1163.

The resulting CMD targeting vector contains a total of approximately 5.3 kb of homology to the heavy chain locus and is designed to generate a mutant mu gene into which has been inserted a neo expression cassette in the unique SmaI site of the first Cmu exon. The targeting vector was linearized with PvuI, which cuts within plasmid sequences, prior to electroporation into ES cells.

Generation and analysis of targeted ES cells. AB-1 ES cells (McMahon, A. P. and Bradley, A. (1990) Cell 62: 1073-1085) were grown on mitotically inactive SNL76/7 cell feeder layers (ibid.), essentially as described in Teratocarcinomas and Embryonic Stem Cells: a Practical Approach, E. J. Robertson, Ed., Oxford: IRL Press, 1987, pp. 71-112. The linearized CMD targeting vector was electroporated into AB-1 cells by the methods described in Hasty et al. (1991) Nature 350: 243-246. Electroporated cells were plated into 100 mm dishes at a density of 1-2×10⁶ cells/dish. After 24 hours, G418 (200 micrograms/ml of active component) and FIAU (5×10⁻⁷ M) were added to the medium, and drug-resistant clones were allowed to develop over 8-9 days. Clones were picked, trypsinized, divided into two portions, and further expanded. Half of the cells derived from each clone were then frozen and the other half analyzed for homologous recombination between vector and target sequences.

DNA analysis was carried out by Southern blot hybridization. DNA was isolated from the clones as described by Laird et al. (1991) Nucleic Acids Res. 19:4293). Isolated genomic DNA was digested with SpeI and probed with a 915 bp SacI fragment, probe A (FIG. 2C), which hybridizes to a sequence between the mu intronic enhancer and the mu switch region. Probe A detects a 9.9 kb SpeI fragment from the wild type locus, and a diagnostic 7.6 kb band from a mu locus which has homologously recombined with the CMD targeting vector (the neo expression cassette contains a SpeI site).

Of 1132 G418 and FIAU resistant clones screened by Southern blot analysis, 3 displayed the 7.6 kb SpeI band indicative of homologous recombination at the mu locus. These 3 clones were further digested with the enzymes BgII, BstXI, and EcoRI to verify that the vector integrated homologously into the mu gene. When hybridized with probe A, Southern blots of wild type DNA digested with BgII, BstXI, or EcoRI produce fragments of 15.7, 7.3, and 12.5 kb, respectively, whereas the presence of a targeted mu allele is indicated by fragments of 7.7, 6.6, and 14.3 kb, respectively. All 3 positive clones detected by the SpeI digest showed the expected BgII, BstXI, and EcoRI restriction fragments diagnostic of insertion of the neo cassette into the Cmu1 exon.

Generation of mice bearing the mutated mu gene. The three targeted ES clones, designated number 264, 272, and 408, were thawed and injected into C57BL/6J blastocysts as described by A. Bradley in Teratocarcinomas and Embryonic Stem Cells: a Practical Approach, E. J. Robertson, Ed., Oxford: IRL Press, 1987, pp. 113-151. Injected blastocysts were transferred into the uteri of pseudopregnant females to generate chimeric mice representing a mixture of cells derived from the input ES cells and the host blastocyst. The extent of ES cell contribution to the chimera can be visually estimated by the amount of agouti coat coloration derived from the ES cell line on the black C57BL/6J background. Clones 272 and 408 produced only low percentage chimeras (i.e. low percentage of agouti pigmentation) but clone 264 produced high percentage male chimeras. These chimeras were bred with C57BL/6J females and agouti offspring were generated, indicative of germline transmission of the ES cell genome. Screening for the targeted mu gene was carried out by Southern blot analysis of BgII digested DNA from tail biopsies (as described above for analysis of ES cell DNA). Approximately 50% of the agouti offspring showed a hybridizing BgII band of 7.7 kb in addition to the wild type band of 15.7 kb, demonstrating a germline transmission of the targeted mu gene.

Analysis of transgenic mice for functional inactivation of mu gene. To determine whether the insertion of the neo cassette into Cmu1 has inactivated the Ig heavy chain gene, a clone 264 chimera was bred with a mouse homozygous for the JHD mutation, which inactivates heavy chain expression as a result of deletion of the JH gene segments (Chen et al. (1993) Immunol. 5: 647-656). Four agouti offspring were generated. Serum was obtained from these animals at the age of 1 month and assayed by ELISA for the presence of murine IgM. Two of the four offspring were completely lacking IgM (Table 1). Genotyping of the four animals by Southern blot analysis of DNA from tail biopsies by BgII digestion and hybridization with probe A (FIG. 2C), and by StuI digestion and hybridization with a 475 bp EcoRI/StuI fragment (ibid.) demonstrated that the animals which fail to express serum IgM are those in which one allele of the heavy chain locus carries the JHD mutation, the other allele the Cmu1 mutation. Mice heterozygous for the JHD mutation display wild type levels of serum Ig. These data demonstrate that the Cmu1 mutation inactivates expression of the mu gene.

Table 1 presents the level of serum IgM, detected by ELISA, for mice carrying both the CMD and JHD mutations (CMD/JHD), for mice heterozygous for the JHD mutation (+/JHD), for wild type (129Sv×C57BL/6J)F1 mice (+/+), and for B cell deficient mice homozygous for the JHD mutation (JHD/JHD).

TABLE 1

| Mouse | Serum IgM (micrograms/ml) | IgH chain genotype |
|---|---|---|
| 42 | <0.002 | CMD/JHD |
| 43 | 196 | +/JHD |
| 44 | <0.002 | CMD/JHD |
| 45 | 174 | +/JHD |
| 129 × BL6 F1 | 153 | +/+ |
| JHD | <0.002 | JHD/JHD |

### EXAMPLE 3

### Generation of Transgenic Mice

The HCo12 human heavy chain transgene. The HCo12 transgene was generated by coinjection of the 80 kb insert of pHC2 (Taylor et al., 1994, Int Immunol., 6: 579-591) and the 25 kb insert of pVx6. The plasmid pVx6 was constructed as described below.

An 8.5 kb HindIII/SaII DNA fragment, comprising the germline human VH1-18 (DP-14) gene together with approximately 2.5 kb of 5′ flanking, and 5 kb of 3′ flanking genomic sequence was subcloned into the plasmid vector pSP72 (Promega, Madison, Wis.) to generate the plasmid p343.7.16. A 7 kb BamHI/HindIII DNA fragment, comprising the germline human VH5-51 (DP-73) gene together with approximately 5 kb of 5′ flanking and 1 kb of 3′ flanking genomic sequence, was cloned into the pBR322 based plasmid cloning vector pGP1f (Taylor et al., 1992, Nucleic Acids Res. 20: 6287-6295), to generate the plasmid p251f.

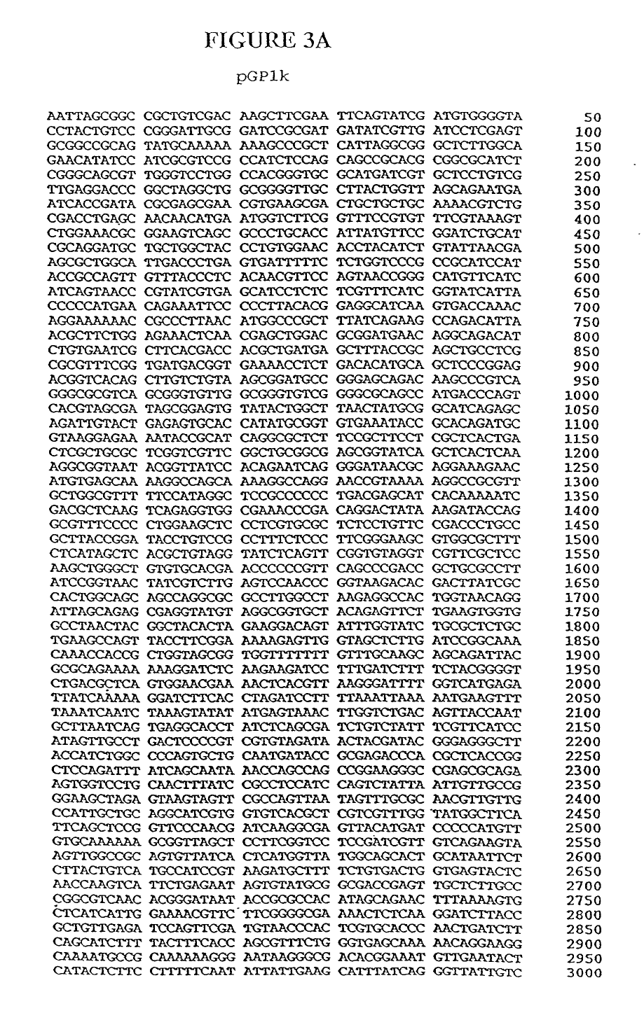

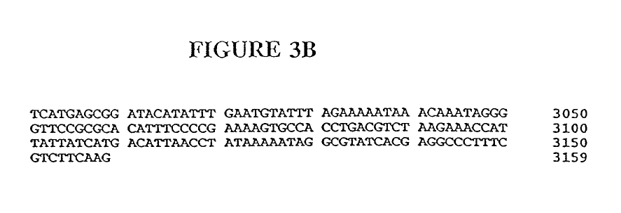

A new cloning vector derived from pGP1f, pGP1k (the sequence of which is presented in FIGS. 3A and 3B), was digested with EcoRV/BamHI, and ligated to a 10 kb EcoRV/BamHI DNA fragment, comprising the germline human VH3-23 (DP47) gene together with approximately 4 kb of 5′ flanking and 5 kb of 3′ flanking genomic sequence. The resulting plasmid, p112.2RR.7, was digested with BamHI/SaII and ligated with the 7 kb purified BamHI/SaII insert of p251f. The resulting plasmid, pVx4, was digested with XhoI and ligated with the 8.5 kb XhoI/SaII insert of p343.7.16. A clone was obtained with the VH1-18 gene in the same orientation as the other two V genes. This clone, designated pVx6, was then digested with NotI and the purified 26 kb insert coinjected, together with the purified 80 kb NotI insert of pHC2 at a 1:1 molar ratio, into the pronuclei of one-half day (C57BL/6J×DBA/2J)F2 embryos as described by Hogan et al. (B. Hogan et al., Manipulating the Mouse Embryo, A Laboratory Manual, 2^{nd} edition, 1994, Cold Spring Harbor Laboratory Press, Plainview N.Y.).

Three independent lines of transgenic mice comprising sequences from both Vx6 and HC2 were established from mice that developed from the injected embryos. These lines are designated (HCo12)14881, (HCo12)15083, and (HCo12)15087. Each of the three lines were then bred with mice comprising the CMD mutation described in Example 2, the JKD mutation (Chen et al., 1993, EMBO J. 12: 811-820), and the (KCo5)9272 transgene (Fishwild et al., 1996, Nature Biotechnology 14: 845-851). The resulting mice express human heavy and kappa light chain transgenes in a background homozygous for disruption of the endogenous mouse heavy and kappa light chain loci.

Additional transgenic mouse strains. Particular strains of mice that may be used to generate IL-4R-reactive monoclonal antibodies are strain ((CMD)++; (JKD)++; (HCo7)11952+/++; (KCo5)9272+/++), and strain ((CMD)++; (JKD)++; (HCo12)15087+/++; (KCo5)9272+/++). Each of these transgenic strains is homozygous for disruptions of the endogenous heavy chain (CMD) and kappa light chain (JKD) loci. Both strains also comprise a human kappa light chain transgene (HCo7), with individual animals either hemizygous or homozygous for insertion #11952. The two strains differ in the human heavy chain transgene used. Mice were hemizygous or homozygous for either the HCo7 or the HCo12 transgene. The CMD mutation is described above in Example 2. The generation of (HCo12)15087 mice is described above (in this example). The JKD mutation (Chen et al., 1993, EMBO J. 12: 811-820) and the (KCo5)9272 (Fishwild et al., 1996, Nature Biotechnology 14: 845-851) and (HCo7)11952 mice, are described in U.S. Pat. No. 5,770,429, which is hereby incorporated by reference.

### EXAMPLE 4

### Generation of Human Anti-IL-4R Monoclonal Antibodies

Transgenic mice. Strain ((CMD)++; (JKD)++; (HCo7)11952+/++; (KCo5)9272+/++which is homozygous for disruptions of the endogenous heavy chain (CMD) and kappa light chain (JKD) loci (see Example 3), was used to generate IL-4R-reactive monoclonal antibodies. This strain also comprises a human kappa light chain transgene (HCo7) with individual animals either hemizygous or homozygous for insertion #11952. Mice were hemizygous or homozygous for the HCo7 transgene. The CMD mutation is described above in Example 2. The JKD mutation (Chen et al., 1993, EMBO J. 12: 811-820) and the (KCo5)9272 (Fishwild et al., 1996, Nature Biotechnology 14: 845-851) and (HCo7)11952 mice, are described in U.S. Pat. No. 5,770,429, which is hereby incorporated by reference.

Immunization. Transgenic mice were initially immunized i.p. with 25 ug IL-4R protein in adjuvant (Titermax, available from Cytrx Corporation, Norcross, Ga.). The immunogen was a human IL-4R polypeptide comprising the extracellular domain of the protein of SEQ ID NO:2. Immunized mice were subsequently boosted every 4 weeks i.p. with the IL-4R immunogen in incomplete Freund's adjuvant. Animals were kept on protocol for 2 to 5 months. Prior to fusion, animals were boosted i.v. on days -4 and -3 with 5 to 8 ug immunogen.

Fusions. Spleen cells harvested from the immunized mice were fused to mouse myeloma cells NS-1 by standard procedures (Harlow and Lane, 1988, Antibodies, A Laboratory Manual, Cold Spring Harbor Laboratory Press, Cold Spring Harbor N.Y.; Kennett et al., 1980, Monoclonal Antibodies, Hybridomas: A New Dimension in Biological Analysis. Plenum, New York; Oi and Hertzenberg, 1980, Immunoglobulin Producing Hybrid Cell Lines, in Selected Methods In Cellular Immunology, ed. Mishell and Shiigi, pp. 357-372. Freeman, San Francisco). Cells were cultured in DMEM, 10% FBS, OPI (Sigma 0-5003), BME (Gibco 21985-023), 3% Origen Hybridoma Cloning Factor (Igen IG50-0615), and 5% P388d1 (ATCC TIB 63) conditioned media. HAT or HT supplement was added to the medium during initial growth and selection.

Hybridoma Screening. To identify hybridomas secreting human antibodies against the IL-4R, ELISA plates (Nunc MaxiSorp) were coated overnight at 4° C. with 100 ul/well human IL-4R at 2.0 ug/ml in PBS. Plates were washed with 100 ul/well PBS-Tween (PBST) containing 1% BSA. Fifty ul cell culture supernatent was added followed by a 1.0 hour incubation. Plates were washed and then incubated for one hour with 100 ul/well goat anti-human IgG conjugated to horseradish peroxidase (Sigma #A-3813, or #A-7164). Plates were washed three times in PBS-Tween between each step.

Wells that read positive by ELISA were screened for their ability to block the binding of IL-4 to IL-4R. ELISA plates were coated overnight with a non-neutralizing mouse anti-human IL-4R antibody M10 at 2 ug/ml. Plates were washed 3X with PBST. 100 ul human IL-4R was added at 10 ng/ml in PBST and incubated for 1.0 hour. Plates were washed 4X with PBST and 100 ul supernatant samples were added and incubated for 1.0 hour. Wells were washed 4X with PBST. 5.0 ng/ml biotinylated IL-4 was added in PBST and incubated for 1.0 hour. 100 ul/well poly80 horseradish peroxidase (RDI) was added at 1:5000 in PBST and incubated for 45 minutes. Plates were washed 5X with PBST, and a colorimetric reagent (3,3′,5,5′ tetramethylbenzidine, available from Kirkegaard and Perry) was added at 100 ul/well until color developed. Reaction was stopped with 100 ul phosphoric acid and plates were read at 450 nm. Absent or reduced signal was interpreted as the antibody binding to receptor in a manner that blocked IL-4 from binding to receptor. Wells that appeared to block binding were expanded and tested for IL-4 and IL-13 blocking in a CD23 expression assay (see Example 5).

### EXAMPLE 5

### Assay for Assessing Blocking Activity

This assay is based on ability of both IL-4 and IL-13 to enhance the expression of the activation-associated surface antigen CD23 on human B cells. Antibodies are tested for the ability to inhibit CD23 expression induced by IL-4 and by IL-13.

Antibodies raised against human IL-4R (huIL-4R) were tested either in the form of hybridoma supernatants or purified protein. Prior to addition to cultures, the antibodies were buffer exchanged against culture medium (RPMI 1640 plus 10% heat-inactivated fetal bovine serum) by centrifugation, using Centricon filter devices (Amicon) with a 10 kDa cutoff.

Human peripheral blood B cells were purified as described previously (Morris et al., J. Biol. Chem. 274:418-423, 1999). The B cells (3×10⁵/well) in culture medium were placed in 96-well round-bottomed microtiter plates and preincubated at room temperature for 30 min with test antibodies at the final concentrations indicated. Recombinant human IL-4 or IL-13 was then added to the cultures at the concentrations indicated, and cells were cultured for 20-24 hours at 37° C. in a humidified atmosphere of 5% CO₂. At the end of the culture period, cells were washed once in PBS+0.02% NaN₃ in the 96-well culture plate and were resuspended in blocking buffer (2% normal rabbit serum +1% normal goat serum in PBS+NaN₃). Phycoerythrin (PE)-conjugated CD23 monoclonal antibody (mAb) or PE-conjugated isotype control mAb (both from Pharmingen) was then added to cells at a final dilution of 1:10. Cells were incubated for 30 minutes at 4° C., washed x3 in PBS+NaN₃ and analyzed on a FacScan (Becton Dickinson) for CD23 expression.

In all experiments, negative controls were included which consisted of cells cultured with hybridoma growth medium or isotype-matched non-blocking human anti-hIL-4R antibody. An anti-huIL-4R murine mAb (R&D Systems), previously shown to block the binding and function of both hIL-4 and hIL-13, was used as a positive control for neutralization of CD23 induction by IL-4 and IL-13.

### EXAMPLE 6

### Hybridoma Cell Line

One hybridoma cell line generated by procedures described above (see Example 4) is designated 6-2. The anti-IL-4R monoclonal antibody secreted by this hybridoma is a blocking antibody, as determined in a conventional plate binding assay, and thus functions as an IL-4 antagonist. The monoclonal antibody produced by 6-2 also exhibits the ability to reduce an IL-13-induced biological activity.

One embodiment of the invention is directed to a hybridoma cell line produced as described above, wherein the hybridoma secretes an isotype IgM mAb directed against human IL-4R. Also provided herein are IgG1 monoclonal antibodies derived from IgM monoclonal antibodies.

All publications, patents and patent applications mentioned in this specification are herein incorporated by reference into the specification to the same extent as if each individual publication, patent or patent application was specifically and individually indicated to be incorporated herein by reference.

Those of skill in the art will recognize, or be able to ascertain using no more than routine experimentation, many equivalents to the specific embodiments of the invention described herein. Such equivalents are intended to be encompassed by the following claims.

What is claimed is:

1. A method for treating renal cancer, comprising administering an IL-4 antagonist to a mammal afflicted with renal cancer.

2. The method of claim 1, wherein the IL-4 antagonist is selected from the group consisting of IL-4 receptors and antibodies that inhibit the binding of IL-4 to an IL-4 receptor.

3. The method of claim 1, wherein the mammal is a human.

4. The method of claim 3, wherein the IL-4 antagonist is a soluble form of the human IL-4 receptor (IL-4R) of SEQ ID NO:2.

5. The method of claim 3, wherein the IL-4 antagonist is an antibody that inhibits the binding of IL-4 to a human IL-4R.

6. The method of claim 5, wherein said antibody is a monoclonal antibody directed against the human IL-4R of SEQ ID NO:2.

7. The method of claim 1, wherein the IL-4 antagonist is administered in the form of a composition that additionally comprises a diluent, excipient, or carrier.

8. A method for treating Burkitt lymphoma, comprising administering an IL-4 antagonist to a mammal afflicted with Burkitt lymphoma.

9. The method of claim 8, wherein the IL-4 antagonist is selected from the group consisting of IL-4 receptors and antibodies that inhibit the binding of IL-4 to an IL-4 receptor.

10. The method of claim 8, wherein the mammal is a human.

11. The method of claim 10, wherein the IL-4 antagonist is a soluble form of the human IL-4 receptor (IL-4R) of SEQ ID NO:2.

12. The method of claim 10, wherein the IL-4 antagonist is an antibody that inhibits the binding of IL-4 to a human IL-4R.

13. The method of claim 12, wherein said antibody is a monoclonal antibody directed against the human IL-4R of SEQ ID NO:2.

14. The method of claim 8, wherein the IL-4 antagonist is administered in the form of a composition that additionally comprises a diluent, excipient, or carrier.

15. A method for treating Hodgkin's Disease, comprising administering an antibody directed against IL-4Rα to a mammal afflicted with Hodgkin's Disease.

16. The method of claim 15, wherein the mammal is a human and the antibody is directed against the human IL-4R of SEQ ID NO:2.

17. The method of claim 16, wherein the antibody is a monoclonal antibody that binds to the extracellular domain of the human IL-4R of SEQ ID NO:2.

18. The method of claim 17, wherein the antibody is a humanized monoclonal antibody or antigen-binding fragment thereof.

19. The method of claim 17, wherein the antibody is a human monoclonal antibody or antigen-binding fragment thereof.

20. A method of reducing proliferation of tumor cells in a Hodgkin's Disease patient, comprising administering an antibody directed against IL-4Rα to the patient.

21. The method for treating non-Hodgkin's lymphoma, comprising administering an antibody directed against IL-4Rα to a mammal afflicted with a non-Hodgkin's lymphoma.

22. The method of claim 21, wherein the mammal is a human and the antibody is directed against the human IL-4R of SEQ ID NO:2.

23. The method of claim 22, wherein the antibody is a monoclonal antibody that binds to the extracellular domain of the human IL-4R of SEQ ID NO:2.

24. The method of claim 23, wherein the antibody is a humanized monoclonal antibody or antigen-binding fragment thereof.

25. The method of claim 23, wherein the antibody is a human monoclonal antibody or antigen-binding fragment thereof.

26. A method for reducing proliferation of non-Hodgkin's lymphoma cells in a patient who has a non-Hodgkin's lymphoma, comprising administering an antibody directed against IL-4Rα to the patient.

27. A method for treating Sezary syndrome, comprising administering an IL-4 antagonist to a mammal afflicted with Sezary syndrome.

28. The method of claim 27, wherein the IL-4 antagonist is selected from the group consisting of IL-4 receptors and antibodies that inhibit the binding of IL-4 to an IL-4 receptor.

29. The method of claim 27, wherein the mammal is a human.

30. A method of claim 29, wherein the IL-4 antagonist is a soluble form of the human IL-4 receptor (IL-4R) of SEQ ID NO:2.

31. The method of claim 29, wherein the IL-4 antagonist is an antibody that inhibits the binding of IL-4 to a human IL-4R.

32. The method of claim 31, wherein said antibody is a monoclonal antibody directed against the human IL-4R of SEQ ID NO:2.

33. The method of claim 27, wherein the IL-4 antagonist is administered in the form of a composition that additionally comprises a diluent, excipient, or carrier.

34. The method of reducing proliferation of tumor cells in Sezary syndrome, comprising administering an antibody directed against IL-4R-alpha.

35. The method of claim 34, wherein the mammal is a human.

36. The method of claim 34, wherein the antibody is directed against the human IL-4R of SEQ ID NO:2.

37. The method of claim 35, wherein the antibody is a monoclonal antibody that binds to the extracellular domain of the human IL-4R of SEQ ID NO:2.

38. The method of claim 36, wherein the antibody is a humanized monoclonal antibody or antigen-binding fragment thereof.

39. The method of claim 36, wherein the antibody is a human monoclonal antibody or antigen-binding fragment thereof.

40. A method for treating cancer, comprising co-administering an IL-4 antagonist and interferon-alpha to a mammal afflicted with cancer.

41. The method of claim 40, wherein the IL-4 antagonist is selected from the group consisting of IL-4 receptors and antibodies that inhibit the binding of IL-4 to an IL-4 receptor.

42. The method of claim 40, wherein the mammal is a human.

43. The method of claim 42, wherein the IL-4 antagonist is a soluble form of the human IL-4 receptor (IL-4R) of SEQ ID NO:2.

44. The method of claim 42, wherein the IL-4 antagonist is an antibody that inhibits the binding of IL-4 to a human IL-4R.

45. The method of claim 44, wherein said antibody is a monoclonal antibody directed against the human IL-4R of SEQ ID NO:2.

46. The method of claim 40, wherein the IL-4 antagonist and interferon-alpha are administered in the form of a composition that additionally comprises a diluent, excipient, or carrier.

47. The method of claim 40, wherein the IL-4 antagonist is co-administered with interferon-alpha in a single formulation.

48. The method of claim 40, wherein the IL-4 antagonist is administered followed by an appropriate time interval and wherein interferon-alpha is subsequently administered.

49. The method of claim 40, wherein the cancer is selected from the group consisting of renal cancer, Burkitt lymphoma, Hodgkin's lymphoma, non-Hodgkin's lymphoma and Sezary syndrome.

50. The method of claim 40, wherein the IL-4 antagonist is administered prior to the administration of interferon-alpha.


SEQUENCE LISTING (3 sequences)

SEQ ID NO: 1 - DNA, 2478 bp, Homo sapiens
  CDS (1)..(2475)
atggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttat

SEQ ID NO: 2 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 3 - protein, 8 aa, Artificial Sequence
  FLAG peptide
DYKDDDDK